(19) United States
(12) Patent Application Publication
(10) Pub. No.: US 2012/0263883 A1
(43) Pub. Date: Oct. 18, 2012

(54) RESIN MODIFIER

(76) Inventors: Hiroaki Kitayama, Wakayama-shi (JP); Shinya Goto, Wakayama-shi (JP); Toshiki Sowa, Wakayama-shi (JP)

(21) Appl. No.: 13/516,577

(22) PCT Filed: Dec. 13, 2010
(86) PCT No.: PCT/JP2010/072330

§ 371 Date: Jun. 15, 2012

(30) Foreign Application Priority Data
Dec. 16, 2009 (JP) 2009-285276
Dec. 16, 2009 (JP) 2009-285283
Dec. 16, 2009 (JP) 2009-285288
Apr. 16, 2010 (JP) 2010-095512
Oct. 5, 2010 (JP) 2010-225876
Oct. 5, 2010 (JP) 2010-225886
Oct. 5, 2010 (JP) 2010-225891

(51) Int. Cl. C07C 309/09; C09D 7/12; C09K 3/16; C08F 2/46

(52) U.S. Cl. 427/487; 106/287.25; 562/36

(57) ABSTRACT

The present invention provides a resin modifier capable of obtaining a coating film having satisfactory basic performances such as antistatic properties, water resistance and transparency, a method for producing a coating film using the resin modifier, and a coating film obtained by the production method. The resin modifier of the present invention is a resin modifier represented by R³¹—O-(AO)ₙ—SO₃⁻, R³²—OSO₃⁻, or R³³—SO₃⁻ and an ammonium ion that has a polymerizable unsaturated group.

### BACKGROUND ART

The present invention relates to a resin modifier, a method for producing a coating film using the resin modifier, and a coating film obtained by the production method.

A resin modifier has hitherto been used so as to improve film characteristics and surface characteristics in the fields of painting, printing and the like.

The below-mentioned Patent Documents 1 to 4 describe antistatic agents having a specific structure for the purpose of enhancing an antistatic effect. For example, Patent Document 1 describes an antistatic agent comprising an anion moiety composed of a specific sulfate and a cation moiety composed of a specific amine. Patent Document 2 describes an antistatic agent comprising an anion moiety composed of a nitric acid ion or an alkylsulfonic acid ion whose alkyl group has 1 to 4 carbon atoms, and a cation moiety composed of a specific amine. Patent Document 3 and Patent Document 4 describe resin compositions including a block (b) which contains 10 to 100 mol % of a quaternary ammonium salt structural unit. The block (b) was added for the purpose of obtaining an antistatic effect. The antistatic agent of Patent Document 1 does not have a polymerizable unsaturated group in the cation moiety. In the antistatic agent of Patent Document 2, the anion moiety is not a sulfate. Furthermore, Patent Documents 5 to 6 describe antistatic resin compositions having excellent scratch resistance or the like.

### PRIOR ART DOCUMENTS

### Patent Documents

Patent Document 1: Japanese Unexamined Patent Publication (Kokai) No. 2007-191684
Patent Document 2: Japanese Unexamined Patent Publication (Kokai) No. 2004-123924
Patent Document 3: Japanese Unexamined Patent Publication (Kokai) No. 11-60855
Patent Document 4: Japanese Unexamined Patent Publication (Kokai) No. 11-60856
Patent Document 5: Japanese Unexamined Patent Publication (Kokai) No. 2009-263627
Patent Document 6: Japanese Unexamined Patent Publication (Kokai) No. 2009-287010

### DISCLOSURE OF THE INVENTION

### Problems to be Solved by the Invention

With the spread of liquid crystal displays (LCD) and the like, a coating film with higher basic performances has recently been required. However, the antistatic agents described in Patent Documents 1 to 4 left a room for improvement in that sufficient antistatic effect has not be obtained yet. It has been earnestly desired to improve not only antistatic properties, but also basic performances such as water resistance and transparency of the obtained coating film.

The present invention provides a resin modifier capable of obtaining a coating film having satisfactory basic performances such as antistatic properties, water resistance and transparency, a method for producing a coating film using the resin modifier, and a coating film obtained by the production method.

### Means for Solving the Problems

The resin modifier of the present invention is a resin modifier represented by any one of the following formulas (Ia), (Ib) and (Ic):

R³¹—O-(AO)ₙ—SO₃⁻B⁺ (Ia)

wherein R³¹ represents a hydrocarbon group having 1 to 22 carbon atoms, AO represents an alkylene oxide group having 2 to 4 carbon atoms, n represents an average addition molar number of AO and is a positive number of 100 or less, and B⁺ represents an ammonium ion (C) having a polymerizable unsaturated group;

R³²—OSO₃⁻B⁺ (Ib)

wherein R³² represents a hydrocarbon group having 6 to 22 carbon atoms, and B+ represents an ammonium ion (C) having a polymerizable unsaturated group; and

R³³—SO₃⁻B⁺ (Ic)

wherein R³³ represents a hydrocarbon group having 8 to 22 carbon atoms, and B+ represents ammonium ion (c) having a polymerizable unsaturated group.

The coating composition of the present invention is a coating composition including the above resin modifier and an organic solvent.

The method for producing a coating film of the present invention is a method for producing a coating film, which includes coating the above coating composition on a base material, and then irradiating the coating composition with active energy rays to form a coating film on the base material.

The coating film of the present invention is a coating film obtained by the above production method.

The coating film of the present invention is a coating film including structures represented by any one of the following formulas (IVa), (IVb), (IVc), (Va), (Vb) and (Vc) in at least one portion:

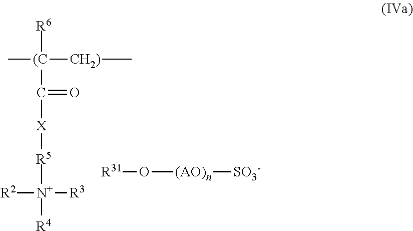

wherein R³¹ represents a hydrocarbon group having 1 to 22 carbon atoms, AO represents an alkylene oxide group having 2 to 4 carbon atoms, n is an average addition molar number of AO and is a positive number of 100 or less, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH;

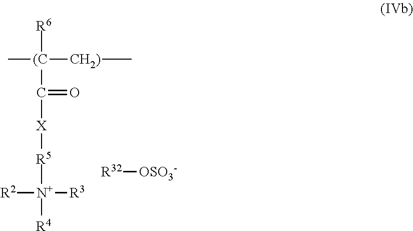

wherein R³² represents a hydrocarbon group having 6 to 22 carbon atoms, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH;

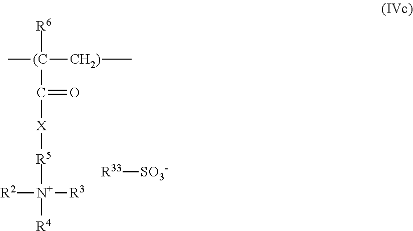

wherein R³³ represents a hydrocarbon group having 8 to 22 carbon atoms, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH;

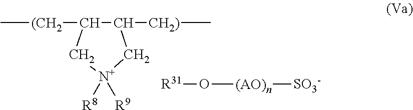

wherein R³¹ represents a hydrocarbon group having 1 to 22 carbon atoms, AO represents an alkylene oxide group having 2 to 4 carbon atoms, n represents an average addition molar number of AO and is a positive number of 100 or less, R⁸ represents a hydrogen atom or a hydrocarbon group having 1 to 8 carbon atoms, and R⁹ represents a hydrocarbon group having 1 to 8 carbon atoms;

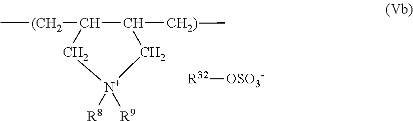

wherein R³² represents a hydrocarbon group having 6 to 22 carbon atoms, R⁸ represents a hydrogen atom or a hydrocarbon group having 1 to 8 carbon atoms, and R⁹ represents a hydrocarbon group having 1 to 8 carbon atoms; and

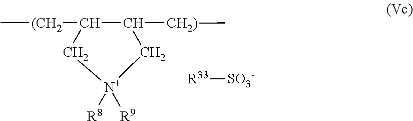

wherein R³³ represents a hydrocarbon group having 8 to 22 carbon atoms, R⁸ represents a hydrogen atom or a hydrocarbon group having 1 to 8 carbon atoms, and R⁹ represents a hydrocarbon group having 1 to 8 carbon atoms.

### Effects of the Invention

According to the resin modifier of the present invention, and, a method for producing a coating film using the coating composition containing the resin modifier, a coating film having excellent basic performances such as antistatic properties, water resistance and transparency can be obtained. The coating film of the present invention has excellent basic performances such as antistatic properties, water resistance and transparency.

### MODE FOR CARRYING OUT THE INVENTION

### Resin Modifier

The resin modifier of the present invention is a resin modifier represented by the above formula (Ia) (hereinafter also referred to as a “compound (Ia)”). The resin modifier of the present invention is also a resin modifier represented by the above formula (Ib) (hereinafter also referred to as a “compound (Ib)”). The resin modifier of the present invention is also a resin modifier represented by the above formula (Ic) (hereinafter also referred to as a “compound (Ic)”).

In the above formula (Ia), from the viewpoint of solubility in organic solvent and industrial availability, R³¹ is a hydrocarbon group having 1 to 22 carbon atoms, preferably a hydrocarbon group having 1 to 20 carbon atoms, more preferably a hydrocarbon group having 8 to 20 carbon atoms, still more preferably a hydrocarbon group having 10 to 18 carbon atoms, and yet more preferably a hydrocarbon group having 12 to 18 carbon atoms. From the viewpoint of antistatic properties, AO is an alkylene oxide group having 2 to 4 carbon atoms, and (AO)ₙ represents addition of ethylene oxide, propylene oxide and butylene oxide alone, and random, block, random/block addition and the like of two or more kinds of alkylene oxide group, and preferably contains ethylene oxide from the viewpoint of antistatic properties and wettability. From the viewpoint of antistatic properties of the compound (Ia), “n” represents an average addition molar number and is a positive number of 100 or less, and “n” is preferably a numeral of 1 to 80, more preferably a numeral of 1 to 70, still more preferably a numeral of 1 to 50, and yet still more preferably a numeral of 1 to 30.

In the above formula (Ib), from the viewpoint of solubility in organic solvent, industrial availability and non-petroleum based material, R³² is a hydrocarbon group having 6 to 22 carbon atoms, preferably a hydrocarbon group having 8 to 22 carbon atoms, more preferably a hydrocarbon group having 8 to 20 carbon atoms, still more preferably a hydrocarbon group having 8 to 18 carbon atoms, and yet still more preferably a hydrocarbon group having 12 to 18 carbon atoms.

In the above formula (Ic), from the viewpoint of solubility in organic solvent and industrial availability, R³³ is a hydrocarbon group having 8 to 22 carbon atoms, preferably a hydrocarbon group having 8 to 20 carbon atoms, more preferably a hydrocarbon group having 8 to 18 carbon atoms, and still more preferably a hydrocarbon group having 8 to 16 carbon atoms.

In the above formulas (Ia), (Ib) and (Ic), the ammonium ion (C) has a polymerizable unsaturated group from the viewpoint of water resistance and transparency. Examples of the polymerizable unsaturated group include a (meth)acryl ester group, a (meth)acrylamide group, an α,β-unsaturated carbonyl ester or amide group such as a maleic acid ester or maleimide group, an α,β-unsaturated nitrile group, an allyl group, a styryl group, a vinyl group, an isopropenyl group and the like. From the viewpoint of antistatic properties, and wettability of a coating film containing a resin modifier of the present invention, an allyl group or an α,β-unsaturated carbonyl group is preferable. Enhancement of the wettability enables suppression of diffused reflection of the coating film and also improvement of anti-fogging effect.

From the viewpoint of antistatic properties, it is preferred that the above ammonium ion (C) is contained in a compound represented by the following formula (II):

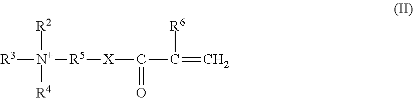

wherein R², R³ and R⁴ each independently represents a hydrogen atom or a hydrocarbon group having 1 to 8 carbon atoms, and is preferably a hydrocarbon group having 1 to 8 carbon atoms from the viewpoint of antistatic properties and industrial availability, and more preferably a hydrocarbon group having 1 or 2 carbon atom. R⁵ represents an alkylene group having 2 to 5 carbon atoms, and is preferably an alkylene group having 2 to 3 carbon atoms from the viewpoint of antistatic properties and industrial availability. R⁶ represents a hydrogen atom or a methyl group, and X represents O or NH and is preferably NH from the viewpoint of antistatic properties. From the viewpoint of antistatic properties and industrial availability, the total number of carbon atoms of R², R³ and R⁴ is preferably from 2 to 12, more preferably from 3 to 9, and still more preferably from 3 to 6.

From the viewpoint of antistatic properties and wettability, it is also preferred that the above ammonium ion (C) is contained in a compound represented by the following formula (III):

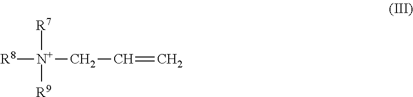

wherein R⁷ and R⁸ each independently represents a hydrogen atom or a hydrocarbon group having 1 to 8 carbon atoms, and is preferably a hydrocarbon group having 1 to 8 carbon atoms from the viewpoint of antistatic properties and industrial availability, more preferably a hydrocarbon group having 1 to 6 carbon atoms, and still more preferably a hydrocarbon group having 1 to 3 carbon atoms. R⁷ is also preferably an allyl group, and R⁸ is also preferably a hydrogen atom or a hydrocarbon group having 1 to 8 carbon atoms, more preferably a hydrocarbon group having 1 to 6 carbon atoms, and still more preferably a hydrocarbon group having 1 to 3 carbon atoms. Specific examples thereof include a methyl group, an ethyl group, a propyl group (an n-propyl group, an iso-propyl group), a butyl group (an n-butyl group, an iso-butyl group, a tert-butyl group), an allyl group, a pentyl group, a hexyl group, an octyl group, a 2-ethylhexyl group and the like.

R⁹ represents a hydrocarbon group having 1 to 8 carbon atoms. From the viewpoint of antistatic properties and industrial availability, it is preferably a hydrocarbon group having 1 to 6 carbon atoms, and more preferably a hydrocarbon group having 1 to 2 carbon atoms. Specific examples thereof include a methyl group, an ethyl group, a propyl group (an n-propyl group, an iso-propyl group), a butyl group (an n-butyl group, an iso-butyl group, a tert-butyl group), a pentyl group, a hexyl group, an octyl group, a 2-ethylhexyl group and the like.

From the viewpoint of antistatic properties and industrial availability, the total number of carbon atoms of R⁷, R⁸ and R⁹ is preferably from 2 to 15, more preferably from 2 to 12, and still more preferably from 2 to 9.

It is preferred that the compounds (Ia), (Ib) and (Ic) have active energy line curability. The active energy line curability means a property in which curing occurs when irradiated with active energy rays such as ultraviolet rays, electron beams, radiations and X-rays, or using an initiator in combination.

Examples of the group capable of curing (reacting) by active energy rays include an α,β-unsaturated carbonyl group, an allyl group, an α,β-unsaturated nitrile group, a styryl group, a vinyl group and an isopropenyl group. From the viewpoint of practical use, active energy rays are preferably ultraviolet rays.

The compounds (Ia), (Ib) and (Ic) are preferably resin modifiers having antistatic properties, namely antistatic agents. Examples of the antistatic agent include antistatic agents which enable a surface resistivity value of a coating film produced by using the antistatic agent to fall within a range of 5×10¹²Ω or less.

In the present invention, the compounds (Ia), (Ib) and (Ic) may be used alone as the resin modifier, or may be used in combination with the other resin modifier with antistatic properties, which has antistatic properties, water resistance, transparency and the like. From the viewpoint of water resistance and transparency, the amount of the compound (Ia), (Ib) or (Ic) is preferably 50 parts by weight or more, more preferably 70 parts by weight or more, and still more preferably 90 parts by weight or more, based on 100 parts by weight of the total of the resin modifiers.

### [Method for Production of Compounds (Ia), (Ib) and (Ic)]

The compound (Ia) can be obtained by a method in which a salt of R³¹—O-(AO)ₙ—SO₃⁻ with an alkali metal and a salt of ammonium having a polymerizable unsaturated group with halogen are subjected to salt exchange, or a method in which R³¹—O-(AO)ₙ—SO₃H is neutralized with an amine having a polymerizable unsaturated group or an ammonium hydroxide having a polymerizable unsaturated group.

The compound (Ib) can be obtained by a method in which a salt of R³²—OSO₃⁻ with an alkali metal and a salt of ammonium having a polymerizable unsaturated group with halogen are subjected to salt exchange, or a method in which R³²—OSO₃H is neutralized with an amine having a polymerizable unsaturated group or an ammonium hydroxide having a polymerizable unsaturated group.

The compound (Ic) can be obtained by a method in which a salt of R³³—SO₃⁻ with an alkali metal and a salt of ammonium having a polymerizable unsaturated group with halogen are subjected to salt exchange, or a method in which R³³—SO₃H is neutralized with an amine having a polymerizable unsaturated group or an ammonium hydroxide having a polymerizable unsaturated group.

In case of producing the compounds (Ia), (Ib) or (Ic), when raw materials containing water are used, water is used in the synthesis or water is contained in the product, a solution of a resin modifier containing the compounds (Ia), (Ib) or (Ic) can be obtained by dissolving in an organic solvent after dehydration, or dehydrating after addition to an organic solvent. When all substituents of an ammonium group of the above ammonium ion (C) are not hydrogens, it is preferred to obtain by the above-mentioned method of performing salt exchange from the viewpoint of industrial availability, handling properties or the like. When at least one of substituents of an ammonium group of the above ammonium ion (C) is hydrogen, it is preferred to obtain a method of neutralization with the above-mentioned amine from the viewpoint of simplification of the step, for example, the dehydration step can be omitted.

Examples of R³¹ in the salt of R³¹—O-(AO)ₙ—SO₃⁻ with an alkali metal include a methyl group, an ethyl group, a propyl group, a butyl group, a pentyl group, a hexyl group, an octyl group, a nonyl group, a decyl group, an undecyl group, a lauryl group, a myristyl group, a cetyl group, a stearyl group, an oleyl group, a behenyl group, a nonylphenyl group and the like. Examples of (AO)ₙ include a polyoxyethylene group, a polyoxypropylene group, a polyoxybutene group, a polyoxyethylenepolyoxypropylene group and the like. Examples of the alkali metal include lithium, sodium and potassium. Specific examples thereof include lithium, sodium and potassium salts of polyoxyethylene methyl ether sulfate; lithium, sodium and potassium salts of polyoxyethylene lauryl ether sulfate; and lithium, sodium and potassium salts of polyoxyethyleneoleyl ether sulfate.

Examples of R³² in the salt of R³²—OSO₃⁻ with an alkali metal include a hexyl group, an octyl group, a nonyl group, a decyl group, an undecyl group, a lauryl group, a myristyl group, a cetyl group, a stearyl group, an oleyl group, a behenyl group and the like. Examples of the alkali metal include lithium, sodium and potassium. Specific examples thereof include lithium, sodium and potassium salts of lauryl sulfate; lithium, sodium and potassium salts of myristyl sulfate; lithium, sodium and potassium salts of decyl sulfate; and lithium, sodium and potassium salts of 2-hexyl-decyl sulfate.

Examples of R³³ in the salt of R³³—SO₃⁻ with an alkali metal include an octyl group, a nonyl group, a decyl group, an undecyl group, a lauryl group, a myristyl group, a cetyl group, a stearyl group, an oleyl group, a behenyl group, a dodecylphenyl group and the like. Examples of the alkali metal include lithium, sodium and potassium. Specific examples include lithium, sodium and potassium salts of decanesulfonate; and lithium, sodium and potassium salts of pentadecanesulfonate.

The salt of ammonium having a polymerizable unsaturated group with halogen is preferably a salt of those corresponding to the chemical formulas (II) and (III) with a halogen, and preferably a chloride from the viewpoint of industrial availability. Examples of the chemical formula (II) include those in which all of R², R³ and R⁴ are methyl groups, those in which two substituents are methyl groups and one substituent is an ethyl group, those in which two substituents are methyl groups and one substituent is a benzyl group and those in which two substituents are ethyl groups and one substituent is a methyl group. Examples of the moiety in which R², R³, R⁴ and N⁺ of the chemical formula (II) have been removed include a 2-(meth)acryloxyethyl group, a 3-(meth)acryloxypropyl group, a 4-(meth)acryloxybutyl group, a 5-(meth)acryloxypentyl group, a 6-(meth)acryloxyhexyl group, a 8-(meth)acryloxyoctyl group, a 4-(meth) acrylamidebutyl group, a 5-(meth)acrylamidepentyl group, a 6-(meth)acrylamidehexyl group, a 8-(meth)acrylamideoctyl group, a 2-(meth)acrylamideethyl group and a 3-(meth)acrylamidepropyl group. Specific examples of the salt of those corresponding to the chemical formula (II) with a halogen include (3-(meth)acrylamidepropyl)trimethylammonium chloride and the like. Examples of those corresponding to the chemical formula (III) include those in which all of R⁷, R⁸ and R⁹ are methyl groups, those in which all of R⁷, R⁸ and R⁹ are ethyl groups, those in which all of R⁷, R⁸ and R⁹ are propyl groups, those in which two substituents are methyl groups and one substituent is an ethyl group, those in which two substituents are methyl groups and one substituent is a propyl group, those in which two substituents are methyl groups and one substituent is a butyl group, those in which two substituents are methyl groups and one substituent is a pentyl group, a hexyl group, an octyl group or a benzyl group, those in which one substituent is an allyl group and two substituents are methyl groups, those in which one substituent is an allyl group and two substituents are ethyl groups, those in which one substituent is an allyl group and two substituents are propyl groups, and those in which one substituent is an allyl group, one substituent is a methyl group and one substituent is an ethyl group, a propyl group, a butyl group, a hexyl group or an octyl group. Specific examples of the salt of those corresponding to the chemical formula (III) with a halogen include allyltrimethylammonium chloride, diallyldimethylammonium chloride and the like. Commercially available products can be used as these salts.

R³¹—O-(AO)ₙ—SO₃H as a precursor of an alkali metal salt can be obtained, for example, by a method in which a compound represented by R³¹—O-(AO)ⁿ—H is sulfonated by reacting with chlorosulfonic acid or sulfuric anhydride (SO₃ gas). R³¹, AO and n in R³¹—O-(AO)ₙ—H are the same as R³¹ AO and n in the above compound (Ia).

Specific examples of R³¹—O-(AO)ₙ—SO₃H used in neutralization of R³¹—O-(AO)ₙ—SO₃H with an amine having a polymerizable unsaturated group include those in which an alkali metal ion moiety of the above-mentioned specific examples of the salt of R³¹—O-(AO)ₙ—SO₃⁻ with an alkali metal is substituted with a hydrogen ion.

R³²—OSO₃H as a precursor of an alkali metal salt can be obtained, for example, by a method in which a compound represented by R³²—OH is sulfated by reacting with chlorosulfonic acid or sulfuric anhydride (SO₃ gas). R³² in R³²—OH is the same as R³² in the above compound (Ib).

Specific examples of R³²—OSO₃H used in neutralization of R³²—OSO₃H with an amine having a polymerizable unsaturated group include those in which an alkali metal ion moiety of specific examples of the salt of R³²—OSO₃⁻ with an alkali metal is substituted with a hydrogen ion.

R³³—SO₃H as a precursor of an alkali metal salt can be obtained, for example, by a reaction of an olefin with SO₃, and examples of the method of directly obtaining a salt include a method of a reaction of an alkyl halide with a sulfite, typified by a Strecker reaction; and a method in which paraffin is reacted with a mixed gas of SO₂ and Cl₂ under ultraviolet irradiation to form a sulfochloride and then the sulfochloride is hydrolyzed in the presence of an alkali, typified by a Reed reaction.

Specific examples of R³³—SO₃H used in neutralization of R³³—SO₃H with an amine having a polymerizable unsaturated group include those in which an alkali metal ion moiety of specific examples of the salt of R³³—SO₃⁻ with an alkali metal is substituted with a hydrogen ion.

Specific examples of the amine having a polymerizable unsaturated group include an amine in which one of hydrocarbon groups having no polymerizability and a halogen have been removed from the above-mentioned specific examples of the salt of an ammonium having a polymerizable unsaturated group with a halogen. Specific examples thereof include allyldimethylamine in which a methyl group as the hydrocarbon group having no polymerizability, and a chloride as the halogen have been removed from allyltrimethylammonium chloride and the like.

Examples of the compound (Ia) include compounds composed of the above-mentioned R³¹—O-(AO)ₙ—SO⁻ and the above-mentioned ammonium having a polymerizable unsaturated group, and specific examples thereof include (meth)acrylamide propyl trimethyl ammonium poly(1-50)oxyethylene methyl ether sulfate, (meth)acrylamide propyl trimethyl ammonium poly(1-50)oxyethylene lauryl ether sulfate, (meth)acrylamide propyl trimethyl ammonium poly(1-50)oxyethylene oleyl ether sulfate, diallyldimethyl ammonium poly(1-50)oxyethylene lauryl ether sulfate, allyldimethyl ammonium poly(1-50)oxyethylene lauryl ether sulfate, maleimidepropylene trimethyl ammonium poly(1-50)oxyethylene lauryl ether sulfate, 2-vinylpyridinium poly(1-50)oxyethylene lauryl ether sulfate, 1-vinylimidazolium poly(1-50)oxyethylene lauryl ether sulfate, styrylmethylene trimethyl ammonium poly(1-50)oxyethylene lauryl ether sulfate and the like. In the present invention, “poly(1-50)oxyethylene” means that an average addition molar number n of an ethylene oxide group is from 1 to 50.

Examples of the compound (Ib) include compounds composed of the above-mentioned R³²—OSO₃⁻ and the above-mentioned ammonium having a polymerizable unsaturated group, and specific examples thereof include (meth)acrylamide propyl trimethyl ammonium lauryl sulfate, (meth)acrylamide propyl trimethyl ammonium decyl sulfate, diallyldimethyl ammonium lauryl sulfate, allyldimethyl ammonium lauryl sulfate, maleimidepropylene trimethyl ammonium lauryl sulfate, 2-vinylpyridinium lauryl sulfate, 1-vinylimidazolium lauryl sulfate, styrylmethylene trimethyl ammonium lauryl sulfate and the like.

Examples of the compound (Ic) include compounds composed of the above-mentioned R³³—SO₃⁻ and the above-mentioned ammonium having a polymerizable unsaturated group, and specific examples thereof include (meth)acrylamide propyl trimethyl ammonium decane sulfonate, (meth)acrylamide propyl trimethyl ammonium pentadecane sulfonate, diallyl dimethyl ammonium decane sulfonate and the like.

There is no particular limitation on the organic solvent used in the production of the compounds (Ia), (Ib) and (Ic). From the viewpoint of solubility of the compounds (Ia), (Ib) and (Ic), an organic solvent having a solubility parameter (SP value described in POLYMER HANDBOOK THIRD EDITION 1989 by John Wiley and Sons, Inc.) of 15.0 to 30.0 (MPa)^{1/2} is preferable, and organic solvent having solubility parameter of 20.0 to 30.0 (MPa)^{1/2} is more preferable. The solubility parameter is shown in parenthesis. Examples of the organic solvent include aliphatic hydrocarbons such as hexane; alcohols such as methanol, ethanol (26.0), isopropyl alcohol (23.5), methoxyethanol, ethoxyethanol, methoxycarbitol and benzyl alcohol (24.8); ketones such as acetone (20.3), methyl ethyl ketone (19.0) and methyl isobutyl ketone (17.2); halogen solvents such as methylene chloride and chloroform; ethers such as diethylether; aromatics such as toluene (18.3) and xylene; esters such as n-butyl acetate (17.4) and n-ethyl acetate (18.6); methylpyrrolidone and dimethyl sulfoxide; and the like. From the viewpoint of solubility, polar solvents such as alcohols, ketones and esters are preferable. In case the other component of the below-mentioned coating composition, for example, a resin monomer is liquid and is also mutually dissolved with the compounds (Ia), (Ib) and (IC), the resin monomer may be used as the organic solvent.

### [Coating Composition]

The coating composition of the present invention preferably contains a resin modifier represented by any one of the above-mentioned formulas (Ia), (Ib) and (Ic), and an organic solvent. The resin modifier is preferably used in a state of being dissolved in the organic solvent. Examples of the organic solvent used in the coating composition preferably include the same as the above-mentioned organic solvents used in the production of the compounds (Ia), (Ib) and (Ic). Suitable range of the solubility parameter (SP value described in POLYMER HANDBOOK THIRD EDITION 1989 by John Wiley and Sons, Inc.) is also the same as that described above, and the organic solvent is preferably an organic solvent having a solubility parameter within a range from 15.0 to 30.0 (MPa)^{1/2}, and more preferably from 20.0 to 30.0 (MPa)^{1/2}. From the viewpoint of solubility of the resin modifier, polar solvents are preferable. Among these solvents, the above-mentioned alcohols, ketones and esters are preferable. On the other hand, in case the resin monomer used in the coating composition is liquid, when the resin monomer dissolves other components containing the resin modifier of the present invention, the resin monomer may be used as the organic solvent from the viewpoint of improvement in handling properties and simplification of the step.

In the coating composition of the present invention, the content of the resin modifier is preferably from 0.5 to 50% by weight, more preferably from 0.5 to 30% by weight, still more preferably from 1 to 25% by weight, and yet still more preferably from 2 to 20% by weight, from the viewpoint of antistatic properties, water resistance and the like.

It is preferred that the coating composition of the present invention further contains a resin or a resin monomer. There is no particular limitation on the resin or resin monomer to be used as long as it is a resin or resin monomer which is suited for use in coating to a base material in the form of a solution prepared using an organic solvent. For example, the resin or resin monomer may be an active energy line-curabile resin or resin monomer, or a thermosetting resin or resin monomer. From the viewpoint of hardness of the coating film and costs, it is preferred to use a resin or resin monomer capable of reacting by irradiation with active energy rays.

The resin or resin monomer capable of reacting by irradiation with active energy rays beams means a resin or resin monomer having a functional group capable of directly causing a curing reaction by irradiation with active energy ray such as ultraviolet ray and electron, or indirectly causing a curing reaction by an action of an initiator.

From the viewpoint of maintaining transparency, the active energy line-curable resin or resin monomer is preferably an acrylic resin or a monomer thereof. From the viewpoint of improving hardness of the coating film, the resin or resin monomer is preferably a resin or resin monomer which has two or more polymerizable functional groups. Examples of the resin or resin monomer, which satisfies both features, include ethylene glycol (meth)acrylate, polyethylene glycol (meth)acrylate, di(meth)acrylates such as pentaerythritol di(meth)acrylate and pentaerythritol di(meth)acrylate monoalkyl ester; tri(meth)acrylates such as trimethylolpropane tri(meth)acrylate and pentaerythritol tri(meth)acrylate; polyfunctional (meth)acrylates such as pentaerythritol tetra(meth)acrylate, dipentaerythritol penta(meth)acrylate, penta(meth)acrylate of dipentaerythrithol EO adduct, and resins formed by polymerization of these monomers.

The content of the resin or resin monomer capable of reacting by irradiation with active energy rays in the coating composition of the present invention is preferably from 20 to 80% by weight, more preferably from 25 to 80% by weight, and still more preferably from 30 to 75% by weight, from the viewpoint of handling properties and costs. Particularly, when using the resin capable of reacting by irradiation with active energy rays and also using an organic solvent in the coating composition (that is, when the resin capable of reacting by irradiation with active energy rays is not used as the organic solvent), the content is preferably within the above range.

In the coating composition of the present invention, the amount of the resin modifier is preferably from 1 to 50 parts by weight, and more preferably from 2 to 30 parts by weight, based on 100 parts by weight of the resin or resin monomer capable of reacting by irradiation with active energy rays. In that case, the content of the resin modifier in the obtained coating film is preferably from 1 to 50 parts by weight, and more preferably from 2 to 30 parts by weight, based on 100 parts by weight the resin or resin monomer capable of reacting by irradiation with active energy rays.

When the resin or resin monomer is not used in the coating composition of the present invention, the content of the resin modifier in the coating composition is preferably from 50% by weight to 100% by weight in the solid component of the coating composition from the viewpoint of antistatic properties and wettability.

The content of the organic solvent is preferably from 10 to 70% by weight, and more preferably from 20 to 60% by weight, and still more preferably from 20 to 55% by weight, in the coating composition of the present invention from the viewpoint of handling properties such as coating. The amount of the organic solvent in the coating composition also includes the amount of an organic solvent to be introduced from a resin modifier solution.

The coating composition of the present invention preferably contains an ionic liquid from the viewpoint of an improvement in antistatic properties under low humidity, in addition to water resistance and transparency.

Herein, the ionic liquid is preferably a compound represented by the following general formula (VI). X⁺ and Y⁻ each does not have a polymerizable unsaturated group, namely a group capable of curing by the above-mentioned active energy rays:

X⁺Y⁻ (VI)

wherein X⁺ represents a cation, and Y⁻ represents an anion.

The molecular weight of the ionic liquid is preferably from 150 to 1,000, and more preferably from 180 to 800, from the viewpoint of antistatic properties under low humidity.

From the viewpoint of antistatic properties under low humidity, the melting point is preferably 100° C. or lower, more preferably 50° C. or lower, and still more preferably 30° C. or lower. In the present invention, the melting point means a melting point measured by “Method for Measurement of Melting Point and Melting Range of Chemical Product” defined in JIS K0064, or a freezing point measured by “Method for Measurement of Freezing Point of Chemical Product” defined in JIS K0065. The melting point was measured in case of a compound which is solid at room temperature (20° C.), while the freezing point was measured in case of a compound which is liquid at room temperature (20° C.), and the obtained value was regarded as the melting point.

From the viewpoint of antistatic properties under low humidity, a cation represented by the above-mentioned X⁺ is more preferably one or more kinds selected from the group consisting of the following formulas (a) to (d):

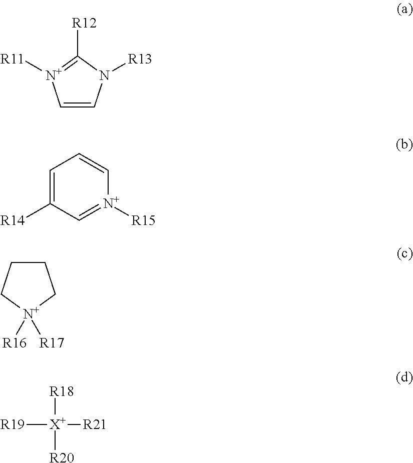

wherein R11 in the formula (a) represents a hydrocarbon group having 1 to 20 carbon atoms, R12 represents a hydrogen atom or a hydrocarbon group having 1 to 20 carbon atoms or a hydroxyl group, R13 represents a hydrocarbon group having 1 to 20 carbon atoms, or a functional group in which one hydrogen of a hydrocarbon group is substituted on a hydroxyl group;
wherein R14 in the formula (b) represents a hydrogen atom or a hydrocarbon group having 1 to 20 carbon atoms or a hydroxyl group, and R15 represents a hydrocarbon group having 1 to 20 carbon atoms, or a functional group in which one hydrogen of a hydrocarbon group is substituted on a hydroxyl group;
wherein R16 and R17 in the formula (c) each independently represents a hydrocarbon group having 1 to 20 carbon atoms, or a functional group in which one hydrogen of a hydrocarbon group is substituted on a hydroxyl group.

Hydrocarbon groups represented by R11 to R17 each independently preferably has 1 to 8 carbon atoms, more preferably 1 to 5 carbon atoms, still yet more preferably 1 to 3 carbon atoms.

wherein X in the formula (d) represents a nitrogen atom, a sulfur atom or a phosphorus atom, R18 represents a hydrogen atom, a hydrocarbon group having 1 to 20 carbon atoms, or a functional group in which one hydrogen of a hydrocarbon group is substituted on a hydroxyl group, R19, R20 and R21 each independently represents a hydrocarbon group having 1 to 20 carbon atoms, or a functional group in which one hydrogen of a hydrocarbon group is substituted on a hydroxyl group, provided that when X is a sulfur atom, R21 is absent.

A hydrocarbon group represented by R18 preferably has 6 to 18 carbon atoms, and more preferably 8 to 18 carbon atoms, and hydrocarbon groups represented by R19 to R21 each independently preferably has 1 to 12 carbon atoms, more preferably 2 to 8 carbon atoms.

From the viewpoint of antistatic properties under low humidity, examples of the anion represented by the above-mentioned Y⁻ include ions of organic acids such as aliphatic (C1-C20) carboxylic acid, fluoroaliphatic (C1-C20) carboxylic acid, poly (average addition molar number: 1-50) oxyalkylene alkyl (C1-C20) ether carboxylic acid, alkyl (C1-C20) sulfuric acid ester, polyoxyalkylene alkyl (C1-C20) sulfuric acid ester, alkane (C1-C20) sulfonic acid, fluoroalkane (C1-C20) sulfonic acid, alkyl (C1-C20) benzenesulfonic acid, alkyl (C1-C20) phosphoric acid ester, poly (average addition molar number: 1-50) oxyalkylene alkyl (C1-C20) phosphoric acid ester, bis(perfluoroalkyl (C1-5) sulfonyl)imide, tris(perfluoroalkyl (C1-C5) sulfonyl)methane and tris(perfluoroalkyl (C1-C5))trifluorophosphate; and ions of inorganic acids such as tetrafluoroboric acid, perchloric acid, hexafluorophosphoric acid, hexafluoroantimonic acid and hexafluoroarsenic acid, phosphoric acid, sulfuric acid, boric acid, halogen, dicyanoamide, tricyanomethide, tetracyanoborate, thiocyanic acid, and dioxalated boric acid.

Specifically, one or more kinds of ionic compounds selected from the group consisting of [CF₃COO⁻], [CH₃SO₄⁻], [C₂H₅SO₄⁻], [C₄H₉SO₄⁻], [C₆H₁₃SO₄⁻], [C₈H₁₇SO₄⁻], [CH₃O(C₂H₄O)ₙSO₃⁻] (n represents an average addition molar number and is from 1 to 5), [CH₃SO₃⁻], [C₂H₅SO₃⁻], [CF₃SO₃⁻], [C₄F₉SO₃⁻], [CH₃C₆H₄SO₃⁻], [N(SO₂CF₃)₂⁻], [(C₂F₅)₃PF₃⁻], [BF₄⁻], [PF₆⁻], [HSO₄⁻], [Cl⁻], [Br⁻], [I⁻], [N(CN)₂⁻], [C(CN)₃⁻], [B(CN)₄⁻], [SCN⁻] and [B(C₂O₄)₂⁻] are preferable from the viewpoint of antistatic properties under low humidity.

Examples of preferable ionic liquid represented by the above-mentioned formula (VI) include the followings.

Examples of the imidazolium compound (compound in which a cation represented by the above-mentioned X⁺ is represented by the formula (a)) include 1-ethyl-3-methylimidazolium tri(pentafluoroethyl)trifluorophosphate (melting point (hereinafter also referred to as “mp”): −1° C.), 1-butyl-3-methylimidazolium dicyanoamide (mp: <−20° C.), 1-butyl-2,3-dimethylimidazolium tetrafluoroborate (mp: 40° C.), 1-butyl-2,3-dimethylimidazolium chloride (mp: 99° C.), 1-butyl-3-methylimidazolium trifluoromethane sulfonate (mp: 17° C.), 1-hexyl-3-methylimidazolium bis(trifluoromethylsulfonyl)imide (mp: −9° C.), 1-butyl-2,3-dimethylimidazolium hexafluorophosphate (mp: 42° C.), 1-ethyl-3-methylimidazolium tetrafluoroborate (mp: 14° C.), 1-ethyl-3-methylimidazolium bromide (mp: 65° C.), 1-hexyl-3-methylimidazolium chloride (mp: <−20° C.), 1-butyl-3-methylimidazolium tetrafluoroborate (mp: <−20° C.), 1-butyl-3-methylimidazolium hexafluorophosphate (mp: 12° C.), 1-ethyl-3-methylimidazolium chloride (mp: 88° C.), 1-methyl-3-octylimidazolium chloride (mp: <−20° C.), 1,3-dimethylimidazolium methyl sulfate (mp: 45° C.), 1-ethyl-3-methylimidazolium trifluoromethane sulfonate (mp: −12° C.), 1-butyl-3-methylimidazolium methyl sulfate (mp: 13° C.), 1-hexyl-3-methylimidazolium tetrafluoroborate (mp: <−20° C.), 1-hexyl-3-methylimidazolium hexafluorophosphate (mp: <−20° C.), 1-hexyl-3-methylimidazolium tri(pentafluoroethyl)trifluorophosphate (mp: −14° C.), 1-butyl-3-methylimidazolium chloride (mp: 73° C.), 1-butyl-3-methylimidazolium bromide (mp: 76° C.), 1-butyl-3-methylimidazolium bis(trifluoromethylsulfonyl)imide (mp: 2° C.), 1-butyl-2,3-dimethylimidazolium trifluoromethane sulfonate (mp: 41° C.), 1-ethyl-3-methylimidazolium trifluoroacetate (mp: <−20° C.), 1-butyl-3-methylimidazolium trifluoroacetate (mp: <−20° C.), 1-ethyl-3-methylimidazolium dicyanamide (mp: <−20° C.), 1-ethyl-3-methylimidazolium methyl sulfate (mp: <−20° C.), 1-ethyl-3-methylimidazolium paratoluenesulfonate (mp: 56° C.), 1-butyl-3-methylimidazolium octyl sulfate (mp: 32° C.), 1-butyl-3-methylimidazolium iodide (mp: <−20° C.), 1-ethyl-3-methylimidazolium bis(trifluoromethylsulfonyl)imide (mp: −15° C.), 1-butyl-2,3-dimethylimidazolium iodide (mp: 97° C.), 1-ethyl-3-methylimidazolium thiocyanate (mp: −20° C.), 1-methyl-3-propylimidazolium iodide (mp: <−20° C.), 1-ethyl-3-methylimidazolium octyl sulfate (mp: 11° C.), 1-butyl-3-methylimidazolium hydrogen sulfate (mp: 38° C.), 1-ethyl-3-methylimidazolium tetracyanoborate (mp: 13° C.), 1-butyl-3-methylimidazolium tri(pentafluoroethyl)trifluorophosphate (mp: 3° C.), 1-decyl-3-methylimidazolium tetracyanoborate (mp: 18° C.), 1-cyanomethyl-3-methylimidazolium bis(trifluoromethylsulfonyl)imide (mp: <−20° C.), 1-(2-hydroxyethyl)-3-methylimidazolium bis(trifluoromethylsulfonyl)imide (mp: <−20° C.), 1-ethyl-3-methylimidazolium methanesulfonate (mp: 35° C.), 1-butyl-3-methylimidazolium methane sulfonate (mp: 75° C.), 1-ethyl-3-methylimidazolium 2(2-methoxyethoxy)ethylsulfate (mp: 15° C.), 1-ethyl-3-methylimidazolium normal butyl sulfate (mp: 24° C.), 1-ethyl-3-methylimidazolium normal hexyl sulfate (mp: 7° C.), 1-butyl-3-methylimidazolium tricyanomethide (mp: <−20° C.), 1-(2-hydroxyethyl)-3-methylimidazolium tri(pentafluoroethyl)trifluorophosphate (mp: <−20° C.), 1-ethyl-3-methylimidazolium iodide (mp: 69° C.), 1-ethyl-3-hydroxyethylimidazolium bromide (mp: <−20° C.), 1-ethyl-3-hydroxypropylimidazolium bromide (mp: <−20° C.) and the like.

Examples of the pyridium compound (compound in which a cation represented by the above-mentioned X⁺ is represented by the formula (b)) include N-butyl-3-methylpyridium tetrafluoroborate (mp: <−20° C.), N-butyl-3-methylpyridium hexafluorophosphate (mp: 46° C.), N-hexyl-4-dimethylamino-pyridium bis(trimethylsulfonyl)imide (mp: <−20° C.), N-(3-hydroxypropyl)pyridium bis(trimethylsulfonyl)imide (mp: <−20° C.), N-ethyl-3-hydroxymethylpyridium ethyl sulfate (mp: <−20° C.), N-ethyl-3-methylpyridium ethyl sulfate (mp: <−20° C.), N-butyl-3-methylpyridium dicyanamide (mp: 16° C.), N-(3-hydroxypropyl)pyridium tri(pentafluoroethyl)trifluorophosphate (mp: <−20° C.) and the like.

Examples of the pyrrolidinium compound (compound in which a cation represented by the above-mentioned X⁺ is represented by the formula (c)) include N-butyl-1-methylpyrrolidinium dicyanoamide (mp: <−20° C.), N-butyl-1-methylpyrrolidinium bis(trifluoromethylsulfonyl)imide (mp: −6° C.), N-butyl-1-methylpyrrolidinium tri(pentafluoroethyl)trifluorophosphate (mp: 4° C.), N-butyl-1-methylpyrrolidinium tetracyanoborate (mp: 22° C.), N-(methoxyethyl)-1-methylpyrrolidinium bis(trimethylsulfonyl)imide (mp: <−20° C.), N-butyl-1-methylpyrrolidinium bis(oxalate(2-)-O,O′)borate (mp: 55° C.), N-(2-methoxyethyl)-1-methylpyrrolidinium tri(pentafluoroethyl)trifluorophosphate (mp: <−20° C.) and the like.

Examples of the ammonium compound and phosphonium compound (compound in which a cation represented by the above-mentioned X⁺ is represented by the formula (d)) include trihexyl(tetradecyl)phosphonium tri(pentafluoroethyl)trifluorophosphate (mp: <−20° C.), trihexyl(tetradecyl)phosphonium bis(trifluoromethylsulfonyl)imide (mp: <20° C.), tetrabutylammonium bis(trifluoromethylsulfonyl)imide (mp: 92° C.), ethyl-dimethyl-propylammonium bis(trifluoromethylsulfonyl)imide (mp: −11° C.), N-ethyl-N,N-dimethyl-2-methoxyethylammonium tri(pentafluoroethyl)trifluorophosphate (mp: <−20° C.), triethylaminenitric acid salt (mp: 12° C.) and the like.

The content of the ionic liquid in the coating composition of the present invention is preferably from 0.3 to 20% by weight, more preferably from 0.5 to 15% by weight, and still more preferably from 1 to 10% by weight, from the viewpoint of antistatic properties under low humidity.

A weight ratio (resin modifier/ionic liquid) of the amount of the resin modifier to the amount of the ionic liquid in the coating composition of the present invention is preferably from 50/50 to 99/1, more preferably from 55/45 to 95/5, and still more preferably from 60/40 to 90/10, from the viewpoint of antistatic properties under low humidity. When the ionic liquid used in the coating composition of the present invention dissolves the other component containing the resin modifier of the present invention, the ionic liquid can be used as the organic solvent from the viewpoint of improvement in handling properties and simplification of the step.

It is also preferred that the coating composition of the present invention contains a conductive polymer from the viewpoint of an improvement in antistatic properties under low humidity, in addition to water resistance and transparency.

The conductive polymer in the present invention refers to a π-conjugated conductive polymer and a δ-conjugated conductive polymer. From the viewpoint of industrial availability, a π-conjugated conductive polymer is preferable. Examples of the π-conjugated conductive polymer include polythiophene, polypyrrole, polyisothianaphthene, polyaniline, polyacetylene, polyparaphenylene, polyphenylenevinylene, polythienylenevinylene, derivatives thereof, and the like. These conductive polymers can be used alone, or two or more kinds can be used in combination.

Among conductive polymers, from the viewpoint of industrial availability, polythiophene, polypyrrole, polyaniline, polyisothianaphthene, and derivatives thereof are preferable, and polythiophene, polypyrrole, polyaniline, and derivatives thereof are more preferably.

Specific examples of the polythiophene derivative include alkyl group-containing polythiophenes such as poly(3-methylthiophene), poly(3-hexylthiophene), poly(3-octylthiophene) and poly(3-dodecylthiophene); ether group-containing polythiophenes such as poly(3-methoxythiophene), poly(3-ethoxythiophene) and poly(3,4-ethylenedioxythiophene); sulfonic acid group-containing polythiophenes such as poly(3-sulfoethylthiophene) and poly(3-sulfobutylthiophene); carboxylic acid-containing polythiophenes such as poly(3-carboxythiophene); and the like.

Specific examples of the polypyrrole derivative include alkyl group-containing polypyrroles such as poly(3-methylpyrrole), poly(3-butylpyrrole), poly(3-decylpyrrole) and poly(3,4-dimethylpyrrole); ether group-containing polypyrroles such as poly(3-methoxypyrrole), poly(3-octoxypyrrole); hydroxy group-containing polypyrroles such as poly(3-hydroxypyrrole); carboxylic acid or carboxylic acid ester group-containing polypyrroles such as poly(3-carboxylpyrrole), poly(3-methyl-4-carboethoxypyrrole) and poly(3-methyl-4-carbobutoxypyrrole); and the like.

Specific examples of the polyisothianaphthene derivative include sulfonic acid group-containing polyisothianaphthenes such as poly(4-sulfoisothianaphthene) and the like.

Specific examples of the polyaniline derivative include alkyl group-containing polyanilines such as poly(2-methylaniline) and poly(2-octylaniline); sulfonic acid group-containing polyanilines such as poly(2-sulfoaniline) and poly(2-sulfo-5-methoxyaniline); and the like.

The weight average molecular weight of the conductive polymer is preferably from 200 to 1,000,000, more preferably from 300 to 500,000, and still more preferably from 500 to 300,000, from the viewpoint of antistatic properties under low humidity.

The content of the conductive polymer in the coating composition of the present invention is preferably from 0.1 to 20% by weight, more preferably from 0.5 to 10% by weight, and still more preferably from 1 to 5% by weight, from the viewpoint of antistatic properties under low humidity and transparency.

A weight ratio (resin modifier/conductive polymer) of the amount of the resin modifier to the amount of the conductive polymer in the coating composition of the present invention is preferably from 30/70 to 99/1, more preferably from 50/50 to 99/1, and still more preferably from 60/40 to 98/2, from the viewpoint of antistatic properties under low humidity.

The conductive polymer may be used in combination with the ionic liquid, or the conductive polymer may be used in place of the ionic liquid.

The coating composition of the present invention can contain water. From the viewpoint of suppressing deterioration of physical properties such as strength and transparency of the obtained coating film, the content of water in the coating composition is preferably less than 5% by weight, more preferably less than 1% by weight, and still more preferably the coating composition does not substantially contain water.

It is possible to mix, in addition to the above-mentioned components, ordinary used initiators, curing agents such as diisocyanate compounds, pigments, dyes, beads such as glass beads, polymer beads and inorganic beads, inorganic fillers such as calcium carbonate and talc, surface modifiers such as leveling agents, and additives such as stabilizers, ultraviolet absorbers and dispersing agents in the coating composition of the present invention.

The coating composition of the present invention preferably contains initiators such as UV initiators and photocation initiators from the viewpoint of acceleration of curing. For example, it is possible to use acetophenones, benzophenones, ketals, anthraquinones, thioxanthones, azo compound, peroxide, 2,3-dialkylsilane compounds, disulfide compounds, thiuram compounds, fluoroamine compound and the like. More specific examples thereof include 1-hydroxy-cyclohexyl-phenyl-ketone, 2-methyl-1[4-(methylthio)phenyl]-2-morpholinopropane-1-one, benzyl dimethyl ketone, 1-(4-dodecylphenyl)-2-hydroxy-2-methylpropane-1-one, 1-(4-isopropylphenyl)-2-hydroxy-2-methylpropane-1-one, benzophenone and the like.

The coating composition of the present invention can be produced by mixing the resin modifier of the present invention and, optionally, other components such as a resin or resin monomer capable of reacting with the coating composition by irradiation with active energy rays, an organic solvent, an ionic liquid, a conductive polymer and an initiator, followed by stirring. There is no particular limitation on mixing order of the respective components. From the viewpoint of solubility of the resin modifier of the present invention, it is preferred that the resin modifier of the present invention is mixed with an organic solvent and then mixed with other components. The temperature in case of mixing is preferably from 0 to 50° C., and more preferably from 5 to 40° C.

### [Method for Producing Coating Film]

The coating film of the present invention is obtained by coating a base material with a coating composition containing the above-mentioned resin modifier of the present invention, optionally drying the coating composition, and then irradiating the coating film with active energy rays. From the viewpoint of hardness of the coating film, it is preferred that the coating composition further contains a resin or a resin monomer.

There is no particular limitation on the base material on which the coating composition of the present invention is applied. Examples of the base material include glass, cellulose-based resins such as triacetate cellulose (TAC) diacetyl cellulose and acetate butylate cellulose, polyester resins such as polyethylene terephthalate (PET), acrylic resin, polyurethane resins, polycarbonate resins, polysulfone resins, polyether resins, polyolefin resins, nitrile resins, polyetherketone resins, polyamide resins and the like.

There is no particular limitation on the coating method, and examples thereof include a bar coating method, a roll coater method, a screen method, a flexo method, a spin coating method, a dip method, a spray method, a slide coating method and the like. After coating, drying is performed under the conditions, for example, a drying temperature within a range from 50 to 150° C. and a drying time within a range from 0.5 to 5 minutes.

From the viewpoint of suppressing damage of a resin base material on which the coating composition is applied, an irradiance of active energy rays is preferably controlled within a range from 10 to 500 mJ in case of using ultraviolet rays as active energy rays.

### [Coating Film]

The coating film of the present invention is a coating film obtained by the above-mentioned production method.

The coating film of the present invention is a coating film including a structure represented by any one of the above formulas (IVa), (IVb), (IVc), (Va), (Vb) and (VC) in at least one portion.

In the formula (IVa), R³¹, R², R³, R⁴, R⁵, R⁶, n and AO are the same as those in the compounds (Ia) and (II). In the formula (IVb), R³², R², R³, R⁴, R⁵ and R⁶ are the same as those in the compounds (Ib) and (II). In the formula (IVc), R³³, R², R³, R⁴, R⁵ and R⁶ are the same as those in the compounds (Ic) and (IT). In the formula (Va), R³¹, R⁸, R⁹, n and AO are the same as those in the compounds (Ia) and (III). In the formula (Vb), R³², R⁸ and R⁹ are the same as those in the compounds (Ib) and (III). In the formula (Vc), R³³, R⁸ and R⁹ are the same as those in the compounds (Ic) and (III).

It is possible to confirm whether structures represented by the above formulas (IVa), (IVb), (IVc), (Va), (Vb) and (Vc) exist in the coating film by extracting the coating film with an organic solvent and analyzing the extract through NMR, IR and the like, or performing mass spectrometry of a surface of the coating film using direct TOF-SIMS.

From the viewpoint of maintaining antistatic properties, a surface resistivity value of the coating film of the present invention is preferably 5×10¹²Ω or less, and more preferably 1×10¹²Ω or less. The surface resistivity value can be measured in accordance with the method described in Examples.

From the viewpoint of maintaining water resistance, the surface resistivity value of the coating film of the present invention after washed with water is preferably 1×10¹³Ω or less, more preferably 5×10¹²Ω or less, and more preferably 1×10¹²Ω or less.

From the viewpoint of transparency, the haze value of the coating film of the present invention is preferably 2% or less, and more preferably 1% or less. The haze value can be measured in accordance with the method described in Examples.

From the viewpoint of maintaining anti-fogging properties, the contact angle of the coating film of the present invention to water is preferably 30 degrees or less. The contact angle can be measured in accordance with the method described in Examples.

The coating film of the present invention can be used for protection of surfaces of various image devices, for example, liquid crystal displays (LCDs), touch panels, plasma display panels (PDPs), electroluminescences (ELs) and optical disks, coating of various lens and the like.

### EXAMPLES

Specific Examples showing the present invention will be described below. Examples 1 to 30 and Comparative Examples 1 to 20 are Examples and Comparative Examples with respect to the compound (Ia). Examples 31 to 55 and Comparative Examples 21 to 39 are Examples and Comparative Examples with respect to the compound (Ib). Examples 56 to 73 and Comparative Examples 40 to 55 are Examples and Comparative Examples with respect to the compound (Ic). Evaluation items in Examples were measured in the following manners.

### [Preparation of Resin Modifier Solution]

In the present Example, first, a resin modifier solution was prepared, and then the thus prepared resin modifier solution, a resin monomer, a curing agent (a UV initiator) and an organic solvent were mixed to produce coating compositions of Examples 1 to 73 and Comparative Examples 1 to 55. With respect to Examples 15 to 25, Examples 44 to 53, Examples 66 to 72, Comparative Examples 12 to 18, Comparative Examples 31 to 36 and Comparative Examples 49 to 53, an ionic liquid was further mixed. With respect to Examples 26 to 30, Examples 54 to 55, Example 73, Comparative Examples 19 to 20, Comparative Examples 37 to 39 and Comparative Examples 54 to 55, a conductive polymer was further mixed. In this case, the resin modifier solution according to the present Example was obtained by salt exchange or neutralization using a specific aqueous sulfate salt solution, or a sulfate composition and a specific ammonium salt or an amine. First, a method for producing an aqueous sulfate salt solution will be described and then a method for preparing a resin modifier solution by salt exchange using the aqueous sulfate salt solution thus produced and an ammonium salt will be described below. Then, a method for preparing a resin modifier solution by neutralization of a sulfate composition as an intermediate for the production of an aqueous sulfate salt solution, and an amine will be described. In Table 1 to Table 3, combinations of raw materials of anion moieties of resin modifiers 1a to 1j, 1m, 1n, 1o, 2a to 2l and 3a to 3f to be produced, and raw materials of cation moieties are shown.

### 1. Production of Aqueous Sulfate Salt Solution

### Production Example 1

An aliphatic alcohol having 12 carbon atoms (manufactured by Kao Corporation under the product name of KALCOL 2098) (500 g) and 0.75 g of KOH were charged in an autoclave equipped with a stirrer, a temperature control device and an automatic introducing device, and then dehydrated at 110° C. under 13 hPa for 30 minutes. After dehydration, replacement by a nitrogen gas was performed and the temperature was raised to 120° C., and then 355 g of ethylene oxide (EO) was charged. After performing an addition reaction and aging at 120° C. over 4 hours, and cooling to 80° C., the unreacted EO was removed under 40 hPa for 30 minutes. After removal of the unreacted EO, 0.8 g of acetic acid was added in the autoclave and the mixture was stirred at 80° C. for 30 minutes, and then extraction was performed to obtain an alkoxylate in which an average EO addition molar number is 3 mol.

The obtained alkoxylate was sulfated by falling-thin film reactor using a SO₃ gas to obtain a poly(3)oxyethylene lauryl ether sulfate composition. The composition (A) (100 g) was neutralized by adding dropwise in 322 g of an aqueous 3.1% by weight NaOH solution to obtain an aqueous sodium poly(3)oxyethylene lauryl ether sulfate solution. The concentration of an active component (% by weight) was measured by the Epton method (JIS K3306). As a result, it was 25% by weight. In the present invention, “poly(3)oxyethylene” means that an average addition molar number n of an ethylene oxide group is 3. The same shall apply hereinafter.

### Production Example 2

In the same manner as in Production Example 1, except that 500 g of an aliphatic alcohol having 12 carbon atoms (manufactured by Kao Corporation under the product name of KALCOL 2098), 0.75 g of KOH, 2,011 g of EO and 0.8 g of acetic acid were used, an aqueous sodium poly(17)oxyethylene lauryl ether sulfate solution (active component of 25% by weight) was obtained.

### Production Example 3

In the same manner as in Production Example 1, except that 300 g of an aliphatic alcohol having 12 carbon atoms (manufactured by Kao Corporation under the product name of KALCOL 2098), 0.45 g of KOH, 3,550 g of EO and 0.48 g of acetic acid were used, an aqueous sodium poly(50)oxyethylene lauryl ether sulfate solution (active component of 25% by weight) was obtained.

### Production Example 4

In the same manner as in Production Example 1, except that 300 g of oleyl alcohol having 18 carbon atoms (reagent, manufactured by Wako Pure Chemical Industries, Ltd.), 0.31 g of KOH, 1,133 g of EO and 0.33 g of acetic acid were used, an aqueous sodium poly(23)oxyethylene oleyl ether sulfate solution (active component of 25% by weight) was obtained.

### Production Example 5

In the same manner as in Production Example 1, except that sulfation and neutralization were performed using 500 g of poly(3)oxyethylene methyl ether (manufactured by NIHON EMULSION Co., Ltd. under the product name of Methyl Tri Glycol) as alkoxylate, an aqueous sodium poly(3)oxyethylene methyl ether sulfate solution was obtained. As the active component of the compound, 52% by weight of a residual component (solid component) subjected to vacuum drying at 70° C. under 200 hPa for 5 hours was used.

### Production Example 6

In the same manner as in Production Example 1, except that 300 g of palmityl alcohol having 16 carbon atoms (manufactured by Kao Corporation under the product name of KALCOL 6098), 0.35 g of KOH, 55 g of EO and 0.38 g of acetic acid were used, an aqueous sodium poly(1)oxyethylene palmityl ether sulfate solution (active component 10% by weight) was obtained.

### Production Example 7

In the same manner as in Production Example 1, except that 300 g of decyl alcohol having 10 carbon atoms (manufactured by Kao Corporation under the product name of KALCOL 1098), 0.53 g of KOH, 167 g of EO and 0.57 g of acetic acid were used, an aqueous sodium poly(2)oxyethylene decyl ether sulfate solution (active component 25% by weight) was obtained.

### Production Example 8

An alcohol having 16 carbon atoms (reagent 2-hexyl-decanol, manufactured by Sigma-Aldrich Corporation) (300 g) was sulfated by a falling-thin film reactor using a SO₂ gas to obtain a 2-hexyl-decyl sulfate composition. The sulfate composition (A) (15 g) was neutralized by adding dropwise in 125 g of an aqueous 1.5% NaOH solution to obtain an aqueous sodium 2-hexyl-decyl sulfate solution. The concentration of the active component (% by weight) was measured by the Epton method (JIS K3306). As a result, it was 25% by weight. This compound was used in the preparation of a resin modifier solution 2d.

### 2. Preparation of Resin Modifier Solution

In accordance with the following methods, resin modifier solutions 1a to 1j, 1m, 1n, 1o, 2a to 2l and 3a to 3f were obtained. Each mixing amount is as shown in Table 1 to Table 3.

### [Production Method 1]

### (Resin Modifier Solution 1a)

The aqueous sodium poly(3)oxyethylene lauryl ether sulfate solution (100 g) obtained in Production Example 1 and 16.4 g of 1-(acrylamidepropyl)trimethylammonium chloride were charged in a recovery flask and the recovery flask was set in a rotary evaporator (ROTARY EBAPORATOR N-1000, manufactured by TOKYO RIKAKIKAI CO., LTD.) and then rotational stirring (rotational speed: SPEED 4) was performed at room temperature (25° C.) under normal pressure (1,013 hPa) for 5 minutes. Then, moisture was removed while blowing leaked air to a sample at 40° C. under 300 hPa and then moisture was removed at 40° C. under 1 hPa for 2 hours. After adding 45 g of isopropyl alcohol in the recovery flask, rotary stirring was performed by a rotary evaporator at room temperature under normal pressure for 30 minutes. The obtained suspension was filtered through a 0.2 μm membrane to obtain a resin modifier solution a (solid component of 43% by weight).

### (Resin Modifier Solutions 1b to 1d, 1f to 1i, 1m, 1n and 1o)

In the same manner as in the preparation of the resin modifier solution a, except that 100 g of the aqueous sodium poly(3)oxyethylene lauryl ether sulfate solution and 16.4 g of 1-(acrylamidepropyl)trimethylammonium chloride were changed to materials in Table 1 and also the use amounts are as shown in Table 1, resin modifier solutions 1b to 1d, 1f to 1i, 1m, 1n and 1o were obtained.

### [Production Method 2]

### (Resin Modifier Solutions 1e)

The poly(3)oxyethylene lauryl ether sulfate composition obtained as the intermediate of Production Example 1 was used as 100% by weight of an active component. In a four-necked flask equipped with a stirrer, charged with 50 g of isopropyl alcohol and 8.8 g of allyldimethylamine, 41.2 g of a poly(3)oxyethylene lauryl ether sulfate composition was added dropwise under a nitrogen gas flow over 20 minutes while ice cooling. Furthermore, aging was performed for 10 minutes to obtain a resin modifier solution 1e. The amount of the residual component (solid component) subjected to vacuum drying at 120° C. under 200 hPa for 5 hours was 50% by weight.

### (Resin Modifier Solution 1j)

In the same manner as in the preparation of the resin modifier solution 1e, except that 8.8 g of allyldimethylamine was changed to 15.4 g of Triethanolamine, a resin modifier solution 1j was obtained.

### [Production Method 3]

### (Resin Modifier Solution 2a)

In a recovery flask, 25 g of sodium lauryl sulfate (reagent, manufactured by Sigma-Aldrich Corporation) was dissolved in 75 g of ion-exchange water. To the solution, 23.9 g of 1-(acrylamidepropyl)trimethylammonium chloride (reagent, manufactured by Tokyo Chemical Industry Co., Ltd.) was added, and the recovery flask was set in a rotary evaporator (ROTARY EBAPORATOR N-1000, manufactured by TOKYO RIKAKIKAI CO., LTD.) and then rotational stirring (rotational speed: SPEED 4) was performed at room temperature (25° C.) under normal pressure (1,013 hPa) for 5 minutes. Then, moisture was removed while blowing leaked air to a sample at 40° C. under 300 hPa and moisture was removed at 40° C. under 300 hPa for 2 hours and then moisture was removed at 40° C. under 1 hPa for 2 hours. After adding 40 g of isopropyl alcohol in the recovery flask, rotary stirring was performed by a rotary evaporator at room temperature under normal pressure for 30 minutes. The obtained suspension was filtered through a 0.2 μm membrane to obtain a resin modifier solution 2a (solid component of 49% by weight).

### (Resin Modifier Solutions 2b to 2e, and 2h to 2l)

In the same manner as in the preparation of the resin modifier solution 2a, except that the aqueous sodium lauryl sulfate solution and 1-(acrylamidepropyl)trimethylammonium chloride were changed to materials in Table 2 and also the use amounts are as shown in Table 2, resin modifier solutions 2b to 2e, and 2h to 2l were obtained.

### [Production Method 4]

### (Resin Modifier Solution 2f)

An alcohol having 12 carbon atoms (manufactured by Kao Corporation under the product name of KALCOL 2098) (300 g) was sulfated by a falling-thin film reactor using a SO₃ gas to obtain a lauryl sulfate composition. In a 100 ml four-necked flask equipped with a stirrer, 3.3 g of dimethylallylamine (reagent, manufactured by Tokyo Chemical Industry Co., Ltd.) and 19.8 g of isopropyl alcohol were charged and then 10 g of the lauryl sulfate composition was added dropwise over 5 minutes under a nitrogen gas flow while stirring at 100 rpm under ice cooling to obtain a resin modifier solution 2f (solid component of 40% by weight).

### (Resin Modifier Solution 2g)

In the same manner as in the preparation of the resin modifier solution 2f, except that 3.3 g of dimethylallylamine was changed to 5.7 g of triethanolamine (reagent, manufactured by Wako Pure Chemical Industries, Ltd.), a resin modifier solution 2g was obtained.

### [Production Method 5]

### (Resin Modifier Solution 3a)

In a recovery flask, 25 g of sodium decane sulfonate (reagent, manufactured by Sigma-Aldrich Corporation) was dissolved in 75 g of ion-exchange water. To the solution, 29.0 g of 1-(acrylamidepropyl)trimethylammonium chloride (reagent, manufactured by Tokyo Chemical Industry Co., Ltd.) was added, and the recovery flask was set in a rotary evaporator (ROTARY EBAPORATOR N-1000, manufactured by TOKYO RIKAKIKAI CO., LTD.) and then rotational stirring (rotational speed: SPEED 4) was performed at room temperature (25° C.) under normal pressure (1,013 hPa) for 5 minutes. Then, moisture was removed while blowing leaked air to a sample at 40° C. under 300 hPa for 2 hours and then moisture was removed at 40° C. under 1 hPa for 2 hours. After adding 40 g of isopropyl alcohol in the recovery flask, rotary stirring was performed by a rotary evaporator at room temperature under normal pressure for 30 minutes. The obtained suspension was filtered through a 0.2 μm membrane to obtain a resin modifier solution 3a (solid component of 46% by weight).

### (Resin Modifier Solutions 3b to 3f)

In the same manner as in the preparation of the resin modifier solution 3a, except that the aqueous sodium decane sulfonate solution and 1-(acrylamidepropyl)trimethylammonium chloride were changed to materials in Table 3 and also the use amounts are as shown in Table 3, resin modifier solutions 3b to 3f were obtained.

### 3. Production of Ionic Liquid

In the present Example, a commercially available reagent was used, except for 1-ethyl-3-hydroxyethylimidazolium bromide.

### Production Example 9

### Production of 1-ethyl-3-hydroxyethylimidazolium bromide

In a four-necked flask equipped with a stirrer, charged with 150 g of 2-bromoethanol (reagent manufactured by Wako Pure Chemical Industries, Ltd.), 173 g of 1-ethylimidazole (reagent, manufactured by Tokyo Chemical Industry Co., Ltd.) was added dropwise over 2 hours while ice cooling under a nitrogen gas flow. After removing ice cooling, the temperature inside the reaction system was returned to room temperature (25° C.). After ten minutes have passed after returning to room temperature, heating was performed by a water bath at 60° C. After heating for 15 hours, the water bath was removed and the temperature was returned to room temperature. The reaction product was transferred to a separatory funnel, washed five times with 500 ml of diethylether and then washed five times with 500 ml of ethyl acetate. The solvent dissolved at 70° C. under 10 hPa was removed from the washed product to obtain 238 g of the objective 1-ethyl-3-hydroxyethylimidazolium bromide.

TABLE 1

| | | Raw compound of resin modifier | | | |
|---|---|---|---|---|---|
| | | Anion moiety | | | |
| | | | Active | | |
| Resin | | | component | Use | |
| modifier | Production | | (% by | amount | Cation moiety |
| solution | method | Compound | weight) | (g) | Compound |
| 1a | 1 | Aqueous sodium | 25 | 100 | 1-(acrylamidepropyl)trimethylammonium |
| | | poly(3)oxyethylene lauryl | | | chloride 1) |
| | | ether sulfate solution | | | |
| 1b | 1 | Aqueous sodium | 25 | 100 | 1-(acrylamidepropyl)trimethylammonium |
| | | poly(17)oxyethylene lauryl | | | chloride 1) |
| | | ether sulfate solution | | | |
| 1c | 1 | Aqueous sodium | 25 | 100 | Diallyldimethylammonium |
| | | poly(17)oxyethylene lauryl | | | choride 2) |
| | | ether sulfate solution | | | |
| 1d | 1 | Aqueous sodium | 25 | 100 | Diallyldimethylammonium |
| | | poly(50)oxyethylene lauryl | | | choride 2) |
| | | ether sulfate solution | | | |
| 1e | 2 | Poly (3) oxyethylene lauryl | 100 | 41.2 | Allyldimethylamine 2) |
| | | ether sulfate composition | | | |
| 1f | 1 | Aqueous sodium | 25 | 100 | 1-(acrylamidepropyl)trimethylammonium |
| | | poly(23)oxyethylene oleyl | | | chloride 1) |
| | | ether sulfate solution | | | |
| 1g | 1 | Aqueous sodium perchlorate | 50 | 19.7 | 1-(acrylamidepropyl)trimethylammonium |
| | | solution 3) | | | chloride 1 |
| 1h | 1 | Aqueous methanesulfonic | 30 | 21.4 | 1-(acrylamidepropyl)trimethylammonium |
| | | acid solution 4) | | | chloride 1) |
| 1i | 1 | Aqueous methanesulfonic | 30 | 21.4 | Diallyldimethylammonium |
| | | acid solution 4) | | | choride 2) |
| 1j | 2 | Poly (3) oxyethylene lauryl | 100 | 41.2 | Triethanolamine 2) |
| | | ether sulfate composition | | | |
| 1m | 1 | Aqueous sodium | 52 | 40 | 1-(acrylamidepropyl)trimethylammonium |
| | | poly(3)oxyethylene methyl | | | chloride 1) |
| | | ether sulfate solution | | | |
| 1n | 1 | Aqueous sodium | 10 | 100 | Diallyldimethylammonium |
| | | poly(1)oxyethylene palmityl | | | choride 2) |
| | | ether sulfate solution | | | |
| 1o | 1 | Aqueous sodium | 25 | 50 | 1-(acrylamidepropyl)trimethylammonium |
| | | poly(2)oxyethylene decyl | | | chloride 1) |
| | | ether sulfate solution | | | |

| | | Raw compound of | | | |
|---|---|---|---|---|---|
| | | resin modifier | | | |
| | | Cation moiety | | Organic | Solid component |
| | | Active | | solvent in | of resin |
| Resin | | component | Use | case of | modifier |
| modifier | Production | (% by | amount | production of | solution |
| solution | method | weight) | (g) | resin modifier | (% by weight) |
| 1a | 1 | 75 | 16.4 | Isopropyl | 43 |
| | | | | alcohol | |
| 1b | 1 | 75 | 6.6 | Isopropyl | 42 |
| | | | | alcohol | |
| 1c | 1 | 65 | 6.0 | Isopropyl | 48 |
| | | | | alcohol | |
| 1d | 1 | 65 | 2.5 | Isopropyl | 48 |
| | | | | alcohol | |
| 1e | 2 | 100 | 8.8 | Isopropyl | 50 |
| | | | | alcohol | |
| 1f | 1 | 75 | 5.2 | Isopropyl | 50 |
| | | | | alcohol | |
| 1g | 1 | 75 | 22.1 | Methanol | 55 |
| 1h | 1 | 75 | 15.0 | Methanol | 26 |
| 1i | 1 | 65 | 13.5 | Isopropyl | 46 |
| | | | | alcohol | |
| 1j | 2 | 100 | 15.4 | Isopropyl | 50 |
| | | | | alcohol | |
| 1m | 1 | 75 | 21.6 | Isopropyl | 51 |
| | | | | alcohol | |
| 1n | 1 | 65 | 6.4 | Isopropyl | 49 |
| | | | | alcohol | |
| 1o | 1 | 75 | 9.9 | Isopropyl | 65 |
| | | | | alcohol | |

1) Reagent manufactured by Tokyo Chemical Industry Co., Ltd.
2) Reagent manufactured by Wako Pure Chemical Industries, Ltd.
3) prepared by diluting sodium perchlorate monohydrate (reagent manufactured by Wako Pure Chemical Industries, Ltd.) with ion-exchange water
4) prepared by diluting methanesulfonic acid (reagent manufactured by Wako Pure Chemical Industries, Ltd.) with ion-exchange water

TABLE 2

| | | Raw compound of resin modifier | | | | | | | Solid |
|---|---|---|---|---|---|---|---|---|---|
| | | Anion moiety | | | Cation moiety | | | Organic | component |
| | | | Active | | | Active | | solvent in | of resin |
| Resin | | | compo- | | | compo- | | case of | modifier |
| modi- | Produc- | | nent | Use | | nent | Use | production | solution |
| fier | tion | | (% by | amount | | (% by | amount | of resin | (% by |
| solution | method | Compound | weight) | (g) | Compound | weight) | (g) | modifier | weight) |
| 2a | 3 | Aqueous sodium lauryl | 25 | 100 | 1-(acrylamidepropyl)trimethylammonium | 75 | 23.9 | Isopropyl | 49 |
| | | sulfate solution1) | | | chloride 2) | | | alcohol | |
| 2b | 3 | Aqueous sodium | 10 | 250 | 1-(acrylamidepropyl)trimethylammonium | 75 | 21 | Isopropyl | 25 |
| | | myristyl sulfate | | | chloride 2) | | | alcohol | |
| | | solution 1) | | | | | | | |
| 2c | 3 | Aqueous sodium decyl | 25 | 50 | 1-(acrylamidepropyl)trimethylammonium | 75 | 13.2 | Isopropyl | 47 |
| | | sulfate solution 1) | | | chloride 2) | | | alcohol | |
| 2d | 3 | Aqueous sodium 2- | 10 | 100 | 1-(acrylamidepropyl)trimethylammonium | 75 | 8.0 | Isopropyl | 28 |
| | | hexyl-decyl sulfate | | | chloride 2) | | | alcohol | |
| | | solution | | | | | | | |
| 2e | 3 | Aqueous sodium decyl | 25 | 100 | Diallyldimethylammonium | 65 | 23.9 | Isopropyl | 50 |
| | | sulfate solution 1) | | | choride 3) | | | alcohol | |
| 2f | 4 | Lauryl sulfate | 100 | 10 | Dimethylallylamine 2) | 14.2 | 23.1 | Isopropyl | 40 |
| | | composition | | | | | | alcohol | |
| 2g | 4 | Lauryl sulfate | 100 | 10 | Triethanolamine 3) | 19.5 | 29.3 | Isopropyl | 40 |
| | | composition | | | | | | alcohol | |
| 2h | 3 | Aqueous sodium | 10 | 100 | 1-(acrylamidepropyl)trimethylammonium | 75 | 14.2 | Isopropyl | 40 |
| | | paratoluene sulfonate | | | chloride 2) | | | alcohol | |
| | | solution 4) | | | | | | | |
| 2i | 3 | Aqueous sodium | 30 | 21.4 | 1-(acrylamidepropyl)trimethylammonium | 75 | 15.0 | Methanol | 26 |
| | | metasulfonate | | | chloride 2) | | | | |
| | | solution 4) | | | | | | | |
| 2j | 3 | Aqueous sodium | 30 | 21.4 | Diallyldimethylammonium | 65 | 13.5 | Isopropyl | 46 |
| | | metasulfonate | | | choride 3) | | | alcohol | |
| | | solution 4) | | | | | | | |
| 2k | 3 | Aqueous sodium | 34 | 60.8 | 1-(acrylamidepropyl)trimethylammonium | 75 | 24.9 | Isopropyl | 50 |
| | | octyl sulfonate | | | chloride 2) | | | alcohol | |
| | | solution 5) | | | | | | | |
| 2l | 3 | Aqueous sodium | 10 | 106.7 | Diallyldimethylammonium | 65 | 7.7 | Isopropyl | 48 |
| | | palmityl sulfonate | | | choride 3) | | | alcohol | |
| | | solution 6) | | | | | | | |

1) prepared by diluting reagent Sigma-Aldrich Corporation with ion-exchange water
2) Reagent manufactured by Tokyo Chemical Industry Co., Ltd.
3) Reagent manufactured by Wako Pure Chemical Industries, Ltd.
4) prepared by diluting reagent manufactured by Wako Pure Chemical Industries, Ltd. with ion-exchange water
5) prepared by diluting reagent manufactured by Kanto Chemical Co., Ltd. with ion-exchange water
6) prepared by diluting reagent manufactured by Wako Pure Chemical Industries, Ltd. with ion-exchange water/ethanol mixed solution (in weight ratio of 3/1)

TABLE 3

| | | | | | | | | Solid |
|---|---|---|---|---|---|---|---|---|
| | Raw compound of resin modifier | | | | | | Organic | component |
| | Anion moiety | | | Cation moiety | | | solvent in | of resin |
| | | Active | | | Active | | case of | modifier |
| Resin | | component | Use | | component | Use | production | solution |
| modifier | | (% by | amount | | (% by | amount | of resin | (% by |
| solution | Compound | weight) | (g) | Compound | weight) | (g) | modifier | weight) |
| 3a | Aqueous sodium | 25 | 100 | 1-(acrylamidepropyl)trimethylammonium | 75 | 29.0 | Isopropyl | 46 |
| | decane sulfate | | | chloride 2) | | | alcohol | |
| | solution 1) | | | | | | | |
| 3b | Aqueous sodium | 20 | 150 | 1-(acrylamidepropyl)trimethylammonium | 75 | 24.7 | Isopropyl | 47 |
| | pentadecane sulfate | | | chloride 2) | | | alcohol | |
| | solution 4) | | | | | | | |
| 3c | Aqueous sodium | 25 | 100 | Diallyldimethylammonium chloride 3) | 65 | 25.0 | Isopropyl | 45 |
| | decane sulfate | | | | | | alcohol | |
| | solution 1) | | | | | | | |
| 3d | Aqueous sodium | 10 | 100 | 1-(acrylamidepropyl)trimethylammonium | 75 | 14.2 | Isopropyl | 40 |
| | paratoluene | | | chloride 2) | | | alcohol | |
| | sulfonate solution 5) | | | | | | | |
| 3e | Aqueous sodium | 30 | 21.4 | 1-(acrylamidepropyl)trimethylammonium | 75 | 15.0 | Methanol | 26 |
| | metanesulfonate | | | chloride 2) | | | | |
| | solution 5) | | | | | | | |
| 3f | Aqueous sodium | 30 | 21.4 | Diallyldimethylammonium choride 3) | 65 | 13.5 | Isopropyl | 46 |
| | metanesulfonate | | | | | | alcohol | |
| | solution 5) | | | | | | | |

1) prepared by diluting reagent Sigma-Aldrich Corporation with ion-exchange water
2) Reagent manufactured by Tokyo Chemical Industry Co., Ltd.
3) Reagent manufactured by Wako Pure Chemical Industries, Ltd.
4) Mersolat H95 (manufactured by LANXESS Corp)
5) prepared by diluting reagent manufactured by Wako Pure Chemical Industries, Ltd. with ion-exchange water

Then, preparation of the coating composition will be described. Table 4 to Table 30 are tables in which the compositions of coating compositions (Examples 1 to 73 and Comparative Examples 1 to 55) and physical properties thereof are shown. In Table 4 to Table 8, Table 11, Table 13 to Table 17, Table 20, Table 22 to Table 26 and Table 29, the resin modifiers 1a to 1j, 1m, 1n, 1o, 2a to 2l and 3a to 3f are resin modifiers produced by combinations shown in Tables 1 to 3.

### [Preparation of Coating Composition]

Coating compositions containing a resin modifier with compositions shown in Table 4 to Table 6, Table 13 to Table 15 and Table 22 to Table 24 were prepared as Examples 1 to 14, Comparative Examples 1 to 10, Examples 31 to 43, Comparative Examples 21 to 29, Examples 56 to 65 and Comparative Examples 40 to 47 by mixing an acrylic resin (dipentaerythritol hexaacrylate (DPHA), manufactured by DAICEL-CYTEC Company LTD.) as a resin capable of reacting by irradiation with active energy rays; Irgacure 184 (manufactured by Ciba Specialty Chemicals Corp.) as a curing agent; an organic solvent; and the above-mentioned resin modifier solution as a resin modifier. With respect to Examples 15 to 25, Comparative Examples 11 to 18, Examples 44 to 53, Comparative Examples 30 to 36, Examples 66 to 72 and Comparative Examples 48 to 53, coating compositions containing a resin modifier with each composition shown in Table 7, Table 8, Table 16, Table 17, Table 25 and Table 26 were prepared by mixing an ionic liquid in addition to the above components. With respect to Examples 26 to 30, Comparative Examples 19 to 20, Examples 54 to 55, Comparative Examples 37 to 39, Example 73 and Comparative Examples 54 to 55, coating compositions containing a resin modifier with the composition shown in Table 11, Table 20 and Table 29 were prepared by mixing a conductive polymer.

Isopropyl alcohol was used as the organic solvent in Examples 1 to 73, Comparative Examples 1 to 4, 7, 8, 10 to 14, 17 to 23, 26 to 32, 35 to 41, 44 to 49 and 52 to 55, and methanol was used in Comparative Examples 5, 6, 9, 15, 16, 24, 25, 33, 34, 42, 43, 50 and 51. Each mixing amount was as shown in Table 4 to Table 8, Table 11, Table 13 to Table 17, Table 20, Table 22 to Table 26 and Table 29. Each mixing amount shown in Table 4 to Table 6, Table 13 to Table 15 and Table 22 to Table 24 shows each number of parts by weight when the total weight (solid component) of a resin capable of reacting by irradiating with active energy rays, a curing agent and an active component of a resin modifier is 100 parts by weight. Each mixing amount shown in Table 7, Table 8, Table 16, Table 17, Table 25 and Table 26 shows each number of parts by weight when the total weight (solid component) of a resin capable of reacting by irradiating with active energy rays, a curing agent, an active component of a resin modifier and an ionic liquid is 100 parts by weight. Each mixing amount shown in Table 11, Table 20 and Table 29 shows each number of parts by weight when the total weight (solid component) of a resin capable of reacting by irradiating with active energy rays, a curing agent, an active component of a resin modifier and a conductive polymer is 100 parts by weight.

### [Formation of Coating Film]

Each of the obtained coating compositions was applied over nearly the whole surface of a cellulose triacetate (TAC) film (10 cm in width×12 cm in length×80 μm in thickness) so that a coating film after UV irradiation has a thickness of 4 μm using a bar coater (gap: 9 to 13 μm) and then dried under the drying conditions shown in Table 4 to Table 6, Table 9, Table 10, Table 12, Table 13 to Table 15, Table 18, Table 19, Table 21, Table 22 to Table 24, Table 27, Table 28 and Table 30. The film after drying was UV irradiated (200 mJ) by a UV irradiation apparatus (HTE-505HA, manufactured by High-Tech Corp., using UV lamp, Model USH-500 MB) under a nitrogen gas flow to obtain a coating film (4 μm in thickness). The coating thickness was measured at three points, for example, an upper portion, a center portion and a lower portion on a center line of a width of a coated surface and an average was used.

### [Surface Resistivity Value (Antistatic Properties) of Coating Film]

With respect to coating films (Examples 1 to 11, Examples 15 to 30, Examples 31 to 40, Examples 44 to 55, Examples 56 to 62, Examples 66 to 73, Comparative Examples 1 to 7, Comparative Examples 11 to 20, Comparative Examples 21 to 26, Comparative Examples 30 to 39, Comparative Examples 40 to 44 and Comparative Examples 48 to 55), a surface resistivity value at the center of the film was measured in a room adjusted to temperature of 25° C. and a relative humidity of 45% by an A-4329 type high resistance meter (manufactured by YHP Manufacturing International Corp.). The smaller the numerical value of the surface resistivity value, the more antistatic properties are excellent.

The results are shown in Table 4, Table 5, Table 9, Table 10, Table 12, Table 13, Table 14, Table 18, Table 19, Table 21, Table 22, Table 23, Table 27, Table 28 and Table 30.

### [Surface Resistivity Value (Low-Humidity Antistatic Properties) of Coating Film]

With respect to coating films (Examples 15 to 30, Examples 44 to 55, Examples 66 to 73, Comparative Examples 11 to 20, Comparative Examples 30 to 39 and Comparative Examples 48 to 55), the measurement was performed in a dry room (manufactured by ITSUWA Co., Ltd.) adjusted to a temperature of 25° C. and a dew pint temperature of −60° C. to −70° C. by the above-mentioned method in a dry room after storage for 72 hours.

The results are shown in Table 9, Table 10, Table 12, Table 18, Table 19, Table 21, Table 27, Table 28 and Table 30.

[Surface Resistivity Value (Water Resistance) of Coating Film after Washing with Water)]

With respect to coating films (Examples 1 to 11, Examples 15 to 30, Examples 31 to 40, Examples 44 to 55, Examples 56 to 62, Examples 66 to 73, Comparative Examples 1 to 7, Comparative Examples 11 to 20, Comparative Examples 21 to 26, Comparative Examples 30 to 39, Comparative Examples 40 to 44 and Comparative Examples 48 to 55), a surface resistivity value was measured after washing with water. The coating film was washed with water under the following conditions. While allowing city water to flow from a faucet of a water pipe having an inner diameter of 14 mm at a flow rate of 10 L/min, a test film was arranged at the location of 10 cm immediately under the faucet so as to vertically pour city water, and then washed with water while moving so as to uniformly pour water on the coating surface for 30 seconds. Then, water on the coating surface was removed by Hyper-Dry Paper Towel manufactured by NIPPON PAPER CRECIA Co., LTD. and then dried at a temperature of 25° C. and a humidity of 45% by sending air until droplets disappear. The smaller the numerical value of the surface resistivity value, the more antistatic properties are excellent.

The results are shown in Table 4, Table 5, Table 9, Table 10, Table 12, Table 13, Table 14, Table 18, Table 19, Table 21, Table 22, Table 23, Table 27, Table 28 and Table 30.

### [Haze Value (Transparency) of Coating Film]

With respect to coating films (Examples 1 to 11, Examples 15 to 30, Examples 31 to 40, Examples 44 to 55, Examples 56 to 62, Examples 66 to 73, Comparative Examples 1 to 7, Comparative Examples 11 to 20, Comparative Examples 21 to 26, Comparative Examples 30 to 39, Comparative Examples 40 to 44 and Comparative Examples 48 to 55), the haze value was determined using a haze meter HM-150 manufactured by MURAKAMI COLOR RESEARCH LABORATORY CO., Ltd. in accordance with JIS K7105 (Test Method of Optical Characteristics of Plastic) (5.5 and 6.4)). Specifically, using Integrating Sphere type Spectrophotometric Transmittnace Meter, a diffusion light transmittance and a total light transmittance were measured and the haze value was expressed by a ratio. The smaller the numerical value of the haze value, the more transparency is excellent.

The results are shown in Table 4, Table 5, Table 9, Table 10, Table 12, Table 13, Table 14, Table 18, Table 19, Table 21, Table 22, Table 23, Table 27, Table 28 and Table 30.

### [Contact Angle of Coating Film]

With respect to coating films (Examples 12 to 14, Comparative Examples 8 to 10, Examples 41 to 43, Comparative Examples 27 to 29, Examples 63 to 65 and Comparative Examples 45 to 47), a contact angle to water was measured using a contact angle meter CA-A manufactured by Kyowa Interface Science Co., Ltd. The smaller the contact angle to water, the more water spreads widely in case water undergoes coagulation. Therefore, diffused reflection does not occur, resulting in obtaining of anti-fogging effect.

The results are shown in Table 6, Table 15 and Table 24.

TABLE 4

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Example 1 | 1a | Poly(3)oxyethylene | 1-(acrylamidepropyl)trimethylammonium | 20 | 75 | 5 |
| | | lauryl ether sulfate | | | | |
| Example 2 | 1b | Poly(17)oxyethylene | 1-(acrylamidepropyl)trimethylammonium | 20 | 75 | 5 |
| | | lauryl ether sulfate | | | | |
| Example 3 | 1f | Poly(2 3)oxyethylene | 1-(acrylamidepropyl)trimethylammonium | 20 | 75 | 5 |
| | | oleyl ether sulfate | | | | |
| Example 4 | 1a | Poly(3)oxyethylene | 1-(acrylamidepropyl)trimethylammonium | 5 | 90 | 5 |
| | | lauryl ether sulfate | | | | |
| Example 5 | 1b | Poly(17)oxyethylene | 1-(acrylamidepropyl)trimethylammonium | 5 | 90 | 5 |
| | | lauryl ether sulfate | | | | |
| Example 6 | 1c | Poly(17)oxyethylene | Diallyldimethylammonium | 5 | 90 | 5 |
| | | lauryl ether sulfate | | | | |
| Example 7 | 1d | Poly(50)oxyethylene | Diallyldimethylammonium | 5 | 90 | 5 |
| | | lauryl ether sulfate | | | | |
| Example 8 | 1e | Poly(3)oxyethylene | Allyldimethylamine | 5 | 90 | 5 |
| | | lauryl ether sulfate | | | | |
| Example 9 | 1a | Poly(3)oxyethylene | 1-(acrylamidepropyl)trimethylammonium | 3 | 92 | 5 |
| | | lauryl ether sulfate | | | | |
| Example | 1m | Poly(3)oxyethylene | 1-(acrylamidepropyl)trimethylammonium | 20 | 75 | 5 |
| 10 | | methyl ether sulfate | | | | |
| Example | 1n | Poly( 1)oxyethylene | Diallyldimethylammonium | 7 | 88 | 5 |
| 11 | | palmityl ether sulfate | | | | |

| | Coating | | | | | | |
|---|---|---|---|---|---|---|---|
| | composition | | | | | | |
| | | Solid | Drying conditions of | | Evaluation of coating film | | |
| | | component | coating film | | Antistatic | Water | |
| | Organic | (% by | Temperature | Time | properties | resistance | Transparency |
| | solvent | weight) | (° C.) | (minute) | (Ω) | (Ω) | (%) |
| Example 1 | IPA 1) | 60 | 70 | 1 | 5.0E+09 | 1.0E+10 | 0.4 |
| Example 2 | IPA | 60 | 70 | 1 | 3.2E+10 | 2.5E+10 | 0.2 |
| Example 3 | IPA | 60 | 70 | 1 | 5.2E+10 | 6.2E+10 | 0.2 |
| Example 4 | IPA | 50 | 100 | 2 | 3.2E+10 | 3.2E+11 | 0.2 |
| Example 5 | IPA | 50 | 100 | 2 | 1.0E+11 | 7.9E+11 | 0.2 |
| Example 6 | IPA | 50 | 100 | 2 | 1.0E+12 | 7.9E+12 | 0.2 |
| Example 7 | IPA | 50 | 100 | 2 | 1.0E+12 | 7.9E+12 | 0.3 |
| Example 8 | IPA | 50 | 100 | 2 | 1.6E+12 | 8.0E+12 | 0.2 |
| Example 9 | IPA | 50 | 100 | 2 | 1.0E+11 | 2.5E+12 | 0.2 |
| Example | IPA | 60 | 70 | 1 | 3.1E+11 | 1.0E+11 | 0.2 |
| 10 | | | | | | | |
| Example | IPA | 60 | 70 | 1 | 3.7E+10 | 2.1E+11 | 0.3 |
| 11 | | | | | | | |

Note 1):
In Table 4, poly(1)oxyethylene, poly(3)oxyethylene, poly(17)oxyethylene, poly(23)oxyethylene and poly(50)oxyethylene mean that average addition molar numbers of an ethylene oxide group are respectively 1, 3, 17, 23 and 50.
1) IPA: Isopropyl alcohol
3) Curing agent: Irgacure 184 (manufactured by Ciba Specialty Chemicals Inc.)
4) DPHA: Dipentaerythritol hexaacrylate

TABLE 5

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Comparative | — | — | — | 0 | 95 | 5 |
| Example 1 | | | | | | |
| Comparative | 1k 5) | Chloride | 1-(acrylamidepropyl)trimethylammonium | 5 | 90 | 5 |
| Example 2 | | | | | | |
| Comparative | 1l 6) | Chloride | Diallyldimethylammonium | 5 | 90 | 5 |
| Example 3 | | | | | | |
| Comparative | 1i | Poly(3)oxyethylene | Triethanolamine | 5 | 90 | 5 |
| Example 4 | | lauryl ether sulfate | | | | |
| Comparative | 1g | Perchloric acid | 1-(acrylamidepropyl)trimethylammonium | 5 | 90 | 5 |
| Example 5 | | | | | | |
| Comparative | 1h | Methanesulfonic acid | 1-(acrylamidepropyl)trimethylammonium | 5 | 90 | 5 |
| Example 6 | | | | | | |
| Comparative | 1i | Methanesulfonic acid | Diallyldimethylammonium | 5 | 90 | 5 |
| Example 7 | | | | | | |

| | Coating | | | | | | |
|---|---|---|---|---|---|---|---|
| | composition | | | | | | |
| | | Solid | Drying conditions | | Evaluation of coating film | | |
| | | component | of coating film | | Antistatic | Water | |
| | Organic | (% by | Temperature | Time | properties | resistance | Transparency |
| | solvent | weight) | (° C.) | (minute) | (Ω) | (Ω) | (%) |
| Comparative | IPA 1) | 50 | 100 | 2 | >1.0E+16 | >1.0E+16 | 0.2 |
| Example 1 | | | | | | | |
| Comparative | IPA | 50 | 100 | 2 | 1.6E+15 | 1.0E+15 | 2.2 |
| Example 2 | | | | | | | |
| Comparative | IPA | 50 | 100 | 2 | 5.0E+14 | 1.0E+15 | 1.2 |
| Example 3 | | | | | | | |
| Comparative | IPA | 50 | 100 | 2 | 1.3E+10 | >1.0E+16 | 1.4 |
| Example 4 | | | | | | | |
| Comparative | MeOH 2) | 50 | 100 | 2 | 2.5E+15 | 2.5E+15 | 1.3 |
| Example 5 | | | | | | | |
| Comparative | MeOH | 50 | 100 | 2 | 1.6E+14 | 1.3E+14 | 4.0 |
| Example 6 | | | | | | | |
| Comparative | IPA | 50 | 100 | 2 | 3.2E+14 | 7.9E+14 | 2.1 |
| Example 7 | | | | | | | |

Note 1):
In Table 5, poly(3)oxyethylene means that an average addition molar number of an ethylene oxide group is 3.
1) IPA: Isopropyl alcohol
2) MeOH: Methanol
3) Curing agent: Irgacure 184 (manufactured by Ciba Specialty Chemicals Inc.)
4) DPHA: Dipentaerythritol hexaacrylate
5) Reagent manufactured by Tokyo Chemical Industry Co., Ltd.
6) Reagent manufactured by Wako Pure Chemical Industries, Ltd.

TABLE 6

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Example 12 | 1a | Poly(3)oxyethylene | 1-(acrylamidepropyl)trimethylammonium | 20 | 75 | 5 |
| | | lauryl ether sulfate | | | | |
| Example 13 | 1b | Poly(17)oxyethylene | 1-(acrylamidepropyl)trimethylammonium | 20 | 75 | 5 |
| | | lauryl ether sulfate | | | | |
| Example 14 | 1c | Poly(17)oxyethylene | Diallyldimethylammonium | 20 | 75 | 5 |
| | | lauryl ether sulfate | | | | |
| Comparative | — | — | — | 0 | 95 | 5 |
| Example 8 | | | | | | |
| Comparative | 1h | Methanesulfonic acid | 1-(acrylamidepropyl)trimethylammonium | 20 | 75 | 5 |
| Example 9 | | | | | | |
| Comparative | 1i | Methanesulfonic acid | Diallyldimethylammonium | 20 | 75 | 5 |
| Example 10 | | | | | | |

| | Coating | | | | |
|---|---|---|---|---|---|
| | composition | | | | |
| | | Solid | Drying conditions of | | |
| | | component | coating film | | |
| | Organic | (% by | Temperature | Time | Contact |
| | solvent | weight) | (° C.) | (minute) | angle (degree) |
| Example 12 | IPA 1) | 60 | 70 | 1 | 27 |
| Example 13 | IPA | 60 | 70 | 1 | 24 |
| Example 14 | IPA | 60 | 70 | 1 | 23 |
| Comparative | IPA | 60 | 70 | 1 | 38 |
| Example 8 | | | | | |
| Comparative | MeOH 2) | 60 | 70 | 1 | 39 |
| Example 9 | | | | | |
| Comparative | IPA | 60 | 70 | 1 | 38 |
| Example 10 | | | | | |

Note 2):
In Table 6, poly(3)oxyethylene and poly(17)oxyethylene mean that average addition molar numbers of an ethylene oxide group are respectively 3 and 17.
1) IPA: Isopropyl alcohol
2) MeOH: Methanol
3) Curing agent: Irgacure 184 (manufactured by Ciba Specialty Chemicals. Inc.)
4) DPHA: Dipentaerythritol hexaacrylate

TABLE 7

| | Coating composition | | | |
|---|---|---|---|---|
| | Resin modifier | | | |
| | Kind | Anion moiety | Cation moiety | Ionic liquid |
| Example 15 | 1a | Poly(3)oxyethylene | 1-(acrylamidepropyl)trimethylammonium | 1-ethyl-3-methylimidazolium |
| | | lauryl ether | | ethyl sulfate 4) |
| | | sulfate | | |
| Example 16 | 1b | Poly(17)oxyethylene | 1-(acrylamidepropyl)trimethylammonium | 1-ethyl-3-methylimidazolium |
| | | lauryl ether | | ethyl sulfate |
| | | sulfate | | |
| Example 17 | 1f | Poly(23)oxyethylene | 1-(acrylamidepropyl)trimethylammonium | 1-ethyl-3-methylimidazolium |
| | | oleyl ether sulfate | | ethyl sulfate |
| Example 18 | 1a | Poly(3)oxyethylene | 1-(acrylamidepropyl)trimethylammonium | 1-ethyl-3-methylimidazolium |
| | | lauryl ether | | thiocyanate 4) |
| | | sulfate | | |
| Example 19 | 1b | Poly(17)oxyethylene | 1-(acrylamidepropyl)trimethylammonium | 1-ethyl-3-methylimidazolium |
| | | lauryl ether | | thiocyanate |
| | | sulfate | | |
| Example 20 | 1c | Poly(17)oxyethylene | Diallyldimethylammonium | 1-ethyl-3-methylimidazolium |
| | | lauryl ether | | chloride 4) |
| | | sulfate | | |
| Example 21 | 1d | Poly(50)oxyethylene | Diallyldimethylammonium | N-ethyl-3-methylpyridium |
| | | lauryl ether | | ethyl sulfate 4) |
| | | sulfate | | |
| Example 22 | 1e | Poly(3)oxyethylene | Allyldimethylamine | trihexyl(tetradecyl)phosphonium |
| | | lauryl ether | | bis |
| | | sulfate | | (trifluoromethylsulfonyl)imide 4) |
| Example 23 | 1a | Poly(3)oxyethylene | 1-(acrylamidepropyl)trimethylammonium | 1-ethyl-3-methylimidazolium |
| | | lauryl ether | | bromide 4) |
| | | sulfate | | |
| Example 24 | 1m | Poly(3)oxyethylene | 1-(acrylamidepropyl)trimethylammonium | 1-ethyl-3-hydroxyethyl- |
| | | methyl ether | | imidazolium bromide |
| | | sulfate | | |
| Example 25 | 1n | Poly(1)oxyethylene | Diallyldimethylammonium | 1-ethyl-3-methylimidazolium |
| | | palmityl ether | | ethyl sulfate 4) |
| | | sulfate | | |

| | Coating composition | | | | | |
|---|---|---|---|---|---|---|
| | Mixing amount (parts by weight) | | | | | |
| | Resin | | | | | Solid |
| | modifier | | | | | component |
| | (Active | | Ionic | Curing | Organic | (% by |
| | component) | DPHA 5) | liquid | agent 3) | solvent | weight) |
| Example 15 | 20 | 70 | 5 | 5 | IPA1) | 60 |
| Example 16 | 20 | 70 | 5 | 5 | IPA | 60 |
| Example 17 | 10 | 80 | 5 | 5 | IPA | 60 |
| Example 18 | 20 | 70 | 5 | 5 | IPA | 50 |
| Example 19 | 20 | 70 | 5 | 5 | IPA | 50 |
| Example 20 | 10 | 80 | 5 | 5 | IPA | 50 |
| Example 21 | 10 | 82 | 3 | 5 | IPA | 50 |
| Example 22 | 10 | 80 | 5 | 5 | IPA | 50 |
| Example 23 | 20 | 72 | 3 | 5 | IPA | 50 |
| Example 24 | 20 | 70 | 5 | 5 | IPA | 60 |
| Example 25 | 10 | 80 | 5 | 5 | IPA | 50 |

Note 1):
In Table 7, poly(1)oxyethylene, poly(3)oxyethylene, poly(17)oxyethylene, poly(23)oxyethylene and poly(50)oxyethylene mean that average addition molar numbers of an ethylene oxide group are respectively 1, 3, 17, 23 and 50.
1) IPA: Isopropyl alcohol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 8

| | Coating composition | | | |
|---|---|---|---|---|
| | Resin modifier | | | |
| | Kind | Anion moiety | Cation moiety | Ionic liquid |
| Comparative | — | — | — | — |
| Example 11 | | | | |
| Comparative | 1k | Chloride | 1- | 1-ethyl-3- |
| Example 12 | | | (acrylamidepropyl)trimethylammonium | methylimidazolium |
| | | | | ethyl sulfate 4) |
| Comparative | 1l | Chloride | Diallyldimethylammonium | 1-ethyl-3- |
| Example 13 | | | | methylimidazolium |
| | | | | ethyl sulfate |
| Comparative | 1j | Poly(3)oxyethylene | Triethanolamine | 1-ethyl-3- |
| Example 14 | | lauryl ether | | methylimidazolium |
| | | sulfate | | thiocyanate 4) |
| Comparative | 1g | Perchloric acid | 1- | 1-ethyl-3- |
| Example 15 | | | (acrylamidepropyl)trimethylammonium | hydroxyethyl |
| | | | | imidazolium bromide |
| Comparative | 1h | Methanesulfonic | 1- | 1-ethyl-3- |
| Example 16 | | acid | (acrylamidepropyl)trimethylammonium | methylimidazolium |
| | | | | thiocyanate |
| Comparative | 1i | Methanesulfonic | Diallyldimethylammonium | 1-ethyl-3- |
| Example 17 | | acid | | methylimidazolium |
| | | | | chloride 4) |
| Comparative | — | — | — | 1-ethyl-3- |
| Example 18 | | | | methylimidazolium |
| | | | | ethyl sulfate |

| | Coating composition | | | | | |
|---|---|---|---|---|---|---|
| | Mixing amount | | | | | |
| | (parts by weight) | | | | | |
| | Resin | | | | | Solid |
| | modifier | | | | | component |
| | (Active | | Ionic | Curing | Organic | (% by |
| | component) | DPHA5) | liquid | agent 3) | solvent | weight) |
| Comparative | 0 | 95 | 0 | 5 | IPA 1) | 50 |
| Example 11 | | | | | | |
| Comparative | 20 | 70 | 5 | 5 | IPA | 50 |
| Example 12 | | | | | | |
| Comparative | 10 | 80 | 5 | 5 | IPA | 50 |
| Example 13 | | | | | | |
| Comparative | 20 | 70 | 5 | 5 | IPA | 50 |
| Example 14 | | | | | | |
| Comparative | 20 | 70 | 5 | 5 | MeOH 2) | 50 |
| Example 15 | | | | | | |
| Comparative | 20 | 70 | 5 | 5 | MeOH | 50 |
| Example 16 | | | | | | |
| Comparative | 20 | 70 | 5 | 5 | IPA | 60 |
| Example 17 | | | | | | |
| Comparative | 0 | 90 | 5 | 5 | IPA | 60 |
| Example 18 | | | | | | |

Note 1):
In Table 8, poly(3)oxyethylene means that an average addition molar number of an ethylene oxide group is 3.
1) IPA: Isopropyl alcohol
2) MeOH: Methanol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 9

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Example 15 | 70 | 1 | 3.5E+08 | 6.9E+09 | 1.0E+10 | 0.3 |
| Example 16 | 70 | 1 | 8.7E+08 | 7.6E+09 | 2.5E+10 | 0.2 |
| Example 17 | 70 | 1 | 3.6E+09 | 3.2E+10 | 6.2E+10 | 0.2 |
| Example 18 | 100 | 2 | 5.1E+08 | 8.3E+09 | 3.2E+11 | 0.2 |
| Example 19 | 100 | 2 | 1.3E+09 | 2.3E+10 | 7.9E+11 | 0.2 |
| Example 20 | 100 | 2 | 3.3E+09 | 8.0E+10 | 6.6E+11 | 0.2 |
| Example 21 | 100 | 2 | 7.8E+09 | 7.6E+10 | 2.3E+11 | 0.3 |
| Example 22 | 100 | 2 | 6.9E+09 | 1.3E+11 | 1.2E+11 | 0.2 |
| Example 23 | 100 | 2 | 6.9E+09 | 8.6E+10 | 4.6E+11 | 0.2 |
| Example 24 | 70 | 1 | 6.8E+08 | 6.9E+09 | 3.6E+10 | 0.2 |
| Example 25 | 70 | 1 | 3.2E+08 | 9.0E+09 | 9.5E+10 | 0.3 |

TABLE 10

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Comparative | 100 | 2 | >1.0E+16 | >1.0E+16 | >1.0E+16 | 0.2 |
| Example 11 | | | | | | |
| Comparative | 100 | 2 | 1.3E+09 | 4.0E+14 | 1.0E+15 | 2.2 |
| Example 12 | | | | | | |
| Comparative | 100 | 2 | 6.9E+09 | 6.9E+14 | 1.0E+15 | 1.2 |
| Example 13 | | | | | | |
| Comparative | 100 | 2 | 3.6E+09 | 4.0E+14 | >1.0E+16 | 1.4 |
| Example 14 | | | | | | |
| Comparative | 100 | 2 | 8.3E+09 | 3.7E+13 | 2.5E+15 | 1.3 |
| Example 15 | | | | | | |
| Comparative | 100 | 2 | 3.0E+09 | 6.0E+11 | 1.3E+14 | 4.9 |
| Example 16 | | | | | | |
| Comparative | 70 | 1 | 2.9E+09 | 3.0E+11 | 1.6E+11 | 16.2 |
| Example 17 | | | | | | |
| Comparative | 70 | 1 | >1.0E+16 | >1.0E+16 | >1.0E+16 | 0.2 |
| Example 18 | | | | | | |

TABLE 11

| | Coating composition | | | | | | | | | |
|---|---|---|---|---|---|---|---|---|---|---|
| | | | | | Mixing amount (parts by weight) | | | | | Solid |
| | | | | | Resin | | | | | com- |
| | | | | | modifier | | Con- | | | ponent |
| | Resin modifier | | | Conductive | (Active | | ductive | Curing | Organic | (% by |
| | Kind | Anion moiety | Cation moiety | polymer | component) | DPHA 4) | polymer | agent 3) | solvent | weight) |
| Example 26 | 1o | Poly(2)oxyethylene | 1-(acrylamidepropyl) | PED-E60 5) | 20 | 74.5 | 0.5 | 5 | IPA 1) | 43 |
| | | decyl ether sulfate | trimethylammonium | | | | | | | |
| Example 27 | 1o | Poly(2)oxyethylene | 1-(acrylamidepropyl) | PED-E60 | 5 | 87 | 3 | 5 | IPA | 30 |
| | | decyl ether sulfate | trimethylammonium | | | | | | | |
| Example 28 | 1n | Poly(1)oxyethylene | Diallyldimethylammonium | PED-E60 | 10 | 84 | 1 | 5 | IPA | 44 |
| | | decyl ether sulfate | | | | | | | | |
| Example 29 | 1o | Poly(2)oxyethylene | 1-(acrylamidepropyl) | PES-E10 6) | 10 | 83.5 | 1.5 | 5 | IPA | 40 |
| | | decyl ether sulfate | trimethylammonium | | | | | | | |
| Example 30 | 1o | Poly(2)oxyethylene | 1-(acrylamidepropyl) | Panipol-T 7) | 5 | 89.5 | 0.5 | 5 | IPA | 38 |
| | | decyl ether sulfate | trimethylammonium | | | | | | | |
| Comparative | — | — | — | PED-E60 | 0 | 94 | 1 | 5 | IPA | 43 |
| Example 19 | | | | | | | | | | |
| Comparative | — | — | — | Panipol-T | 0 | 90 | 5 | 5 | IPA | 42 |
| Example 20 | | | | | | | | | | |

Note 1):
In Table 11, poly(1)oxyethylene and poly(2)oxyethylene mean that average addition molar numbers of an alkylene oxide group are respectively 1 and 2.
1) IPA: Isopropyl alcohol
3) Curing agent: Irgacure 184 (manufactured by Ciba Specialty Chemicals Inc.)
4) DPHA: Dipentaerythritol hexaacrylate
5) PED-E60: Polythiophene manufactured by Polymerits Corp. Ltd.
6) PES-E10: Polypyrrol manufactured by Polymerits Corp.
7) Panipol-T: Polyaniline manufactured by PANIPOL Oy

TABLE 12

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Example 26 | 70 | 1 | 9.0E+09 | 6.0E+10 | 2.2E+10 | 0.3 |
| Example 27 | 70 | 1 | 5.0E+09 | 1.0E+10 | 1.2E+10 | 0.4 |
| Example 28 | 70 | 1 | 6.6E+09 | 2.0E+10 | 9.0E+09 | 0.3 |
| Example 29 | 70 | 1 | 8.5E+09 | 3.8E+10 | 2.3E+10 | 0.5 |
| Example 30 | 70 | 1 | 2.5E+10 | 9.0E+10 | 7.8E+10 | 0.4 |
| Comparative | 70 | 1 | 8.4E+15 | >1.0E+16 | >1.0E+16 | 0.4 |
| Example 19 | | | | | | |
| Comparative | 70 | 1 | >1.0E+16 | >1.0E+16 | >1.0E+16 | 1.0 |
| Example 20 | | | | | | |

TABLE 13

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Example | 2a | Lauryl sulfate | 1-(acrylamidepropyl)trimethylammonium | 20 | 75 | 5 |
| 31 | | | | | | |
| Example | 2a | Lauryl sulfate | 1-(acrylamidepropyl)trimethylammonium | 5 | 90 | 5 |
| 32 | | | | | | |
| Example | 2b | Myristyl sulfate | 1-(acrylamidepropyl)trimethylammonium | 5 | 90 | 5 |
| 33 | | | | | | |
| Example | 2c | Decyl sulfate | 1-(acrylamidepropyl)trimethylammonium | 20 | 75 | 5 |
| 34 | | | | | | |
| Example | 2c | Decyl sulfate | 1-(acrylamidepropyl)trimethylammonium | 3 | 92 | 5 |
| 35 | | | | | | |
| Example | 2e | Decyl sulfate | Diallyldimethylammonium | 5 | 90 | 5 |
| 36 | | | | | | |
| Example | 2d | 2-hexyldecyl sulfate | 1-(acrylamidepropyl)trimethylammonium | 5 | 90 | 5 |
| 37 | | | | | | |
| Example | 2f | Lauryl sulfate | Dimethylamine | 5 | 90 | 5 |
| 38 | | | | | | |
| Example | 2k | Octyl sulfate | 1-(acrylamidepropyl)trimethylammonium | 20 | 75 | 5 |
| 39 | | | | | | |
| Example | 21 | Palmityl sulfate | Diallyldimethylammonium | 10 | 85 | 5 |
| 40 | | | | | | |

| | Coating | | | | | | |
|---|---|---|---|---|---|---|---|
| | composition | | | | | | |
| | | Solid | Drying conditions of | | Evaluation of coating film | | |
| | | component | coating film | | Antistatic | Water | |
| | Organic | (% by | Temperature | Time | properties | resistance | Transparency |
| | solvent | weight) | (° C.) | (minute) | (Ω) | (Ω) | (%) |
| Example | IPA 1) | 60 | 70 | 1 | 1.3E+10 | 2.0E+10 | 1.0 |
| 31 | | | | | | | |
| Example | IPA | 50 | 100 | 2 | 3.2E+10 | 1.6E+10 | 0.2 |
| 32 | | | | | | | |
| Example | IPA | 50 | 100 | 2 | 4.0E+10 | 5.0E+10 | 0.3 |
| 33 | | | | | | | |
| Example | IPA | 60 | 70 | 1 | 1.6E+09 | 5.0E+09 | 0.5 |
| 34 | | | | | | | |
| Example | IPA | 50 | 100 | 2 | 3.2E+10 | 2.0E+11 | 0.2 |
| 35 | | | | | | | |
| Example | IPA | 50 | 100 | 2 | 1.0E+11 | 4.0E+11 | 0.3 |
| 36 | | | | | | | |
| Example | IPA | 50 | 70 | 1 | 1.6E+10 | 1.3E+11 | 0.4 |
| 37 | | | | | | | |
| Example | IPA | 50 | 100 | 2 | 1.6E+11 | 6.3E+11 | 0.3 |
| 38 | | | | | | | |
| Example | IPA | 45 | 70 | 1 | 1.6E+10 | 2.0E+10 | 0.2 |
| 39 | | | | | | | |
| Example | IPA | 45 | 70 | 1 | 7.9E+10 | 3.2E+11 | 0.4 |
| 40 | | | | | | | |

1) IPA: Isopropyl alcohol
3) Curing agent: Irgacure 184 (manufactured by Ciba Specialty Chemicals Inc.)
4) DPHA: Dipentaerythritol hexaacrylate

TABLE 14

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Comparative | — | — | — | 0 | 95 | 5 |
| Example 21 | | | | | | |
| Comparative | 2g | Lauryl sulfate | Triethanolamine | 20 | 75 | 5 |
| Example 22 | | | | | | |
| Comparative | 2h | Paratoluenesulfonic | 1-(acrylamidepropyl)trimethylammonium | 20 | 75 | 5 |
| Example 23 | | acid | | | | |
| Comparative | 2i | Methanesulfonic | 1-(acrylamidepropyl)trimethylammonium | 20 | 75 | 5 |
| Example 24 | | acid | | | | |
| Comparative | 2i | Methanesulfonic | 1-(acrylamidepropyl)trimethylammonium | 5 | 90 | 5 |
| Example 25 | | acid | | | | |
| Comparative | 2j | Methanesulfonic | Diallyldimethylammonium | 20 | 75 | 5 |
| Example 26 | | acid | | | | |

| | Coating | | | | | | |
|---|---|---|---|---|---|---|---|
| | composition | | | | | | |
| | | Solid | Drying conditions | | Evaluation of coating film | | |
| | | component | of coating film | | Antistatic | Water | |
| | Organic | (% by | Temperature | Time | properties | resistance | Transparency |
| | solvent | weight) | (° C.) | (minute) | (Ω) | (Ω) | (%) |
| Comparative | IPA 1) | 50 | 100 | 2 | >1.0E+16 | >1.0E+16 | 0.2 |
| Example 21 | | | | | | | |
| Comparative | IPA | 60 | 70 | 1 | 1.0E+10 | 1.0E+14 | 13.5 |
| Example 22 | | | | | | | |
| Comparative | IPA | 60 | 70 | 1 | 6.3E+11 | 3.2E+12 | 3.0 |
| Example 23 | | | | | | | |
| Comparative | MeOH 2) | 60 | 70 | 1 | 6.3E+12 | 6.3E+13 | 9.2 |
| Example 24 | | | | | | | |
| Comparative | MeOH | 50 | 100 | 2 | 1.6E+14 | 1.3E+14 | 4.0 |
| Example 25 | | | | | | | |
| Comparative | IPA | 60 | 70 | 1 | 1.6E+10 | 1.6E+11 | 15.0 |
| Example 26 | | | | | | | |

1) IPA: Isopropyl alcohol
2) MeOH: Methanol
3) Curing agent: Irgacure 184 (manufactured by Ciba Specialty Chemicals Inc.)
4) DPHA: Dipentaerythritol hexaacrylate

TABLE 15

| | Coating composition | | | | | |
|---|---|---|---|---|---|---|
| | | | | Mixing amount | | |
| | | | | (parts by weight) | | |
| | | | | Resin | | |
| | | | | modifier | | |
| | Resin modifier | | | (active | | Curing |
| | Kind | Anion moiety | Cation moiety | component) | DPHA 4) | agent 3) |
| Example 41 | 2a | Lauryl sulfate | 1-(acrylamidepropyl)trimethylammonium | 20 | 75 | 5 |
| Example 42 | 2b | Myristyl sulfate | 1-(acrylamidepropyl)trimethylammonium | 20 | 75 | 5 |
| Example 43 | 2e | Decyl sulfate | Diallyldimethylammonium | 20 | 75 | 5 |
| Comparative | — | — | — | 0 | 95 | 5 |
| Example 27 | | | | | | |
| Comparative | 2i | Methanesulfonic acid | 1-(acrylamidepropyl)trimethylammonium | 20 | 75 | 5 |
| Example 28 | | | | | | |
| Comparative | 2h | Paratoluenesulfonic | 1-(acrylamidepropyl)trimethylammonium | 20 | 75 | 5 |
| Example 29 | | acid | | | | |

| | Coating | | | | |
|---|---|---|---|---|---|
| | composition | | | | |
| | | Solid | Drying conditions of | | |
| | | component | coating film | | |
| | Organic | (% by | Temperature | Time | Contact |
| | solvent | weight) | (° C.) | (minute) | angle (degree) |
| Example 41 | IPA 1) | 60 | 70 | 1 | 27 |
| Example 42 | IPA | 60 | 70 | 1 | 27 |
| Example 43 | IPA | 60 | 70 | 1 | 23 |
| Comparative | IPA | 60 | 70 | 1 | 38 |
| Example 27 | | | | | |
| Comparative | IPA | 60 | 70 | 1 | 39 |
| Example 28 | | | | | |
| Comparative | IPA | 60 | 70 | 1 | 38 |
| Example 29 | | | | | |

1) IPA: Isopropyl alcohol
3) Curing agent: Irgacure 184 (manufactured by Ciba Specialty Chemicals Inc.)
4) DPHA: Dipentaerythritol hexaacrylate

TABLE 16

| | Coating composition | | | | | | | | | |
|---|---|---|---|---|---|---|---|---|---|---|
| | | | | | Mixing amount | | | | | |
| | | | | | (parts by weight) | | | | | |
| | | | | | Resin | | | | | Solid |
| | | | | | modifier | | | | | component |
| | Resin modifier | | | | (Active | | Ionic | Curing | Organic | (% by |
| | Kind | Anion moiety | Cation moiety | Ionic liquid | component) | DPHA 5) | liquid | agent 3) | solvent | weight) |
| Example | 2a | Lauryl sulfate | 1-(acrylamidepropyl) | 1-ethyl-3- | 20 | 70 | 5 | 5 | IPA 1) | 60 |
| 44 | | | trimethylammonium | methylimidazolium | | | | | | |
| | | | | ethyl sulfate 4) | | | | | | |
| Example | 2a | Lauryl sulfate | 1-(acrylamidepropyl) | 1-ethyl-3- | 10 | 80 | 5 | 5 | IPA | 50 |
| 45 | | | trimethylammonium | methylimidazolium | | | | | | |
| | | | | ethyl sulfate | | | | | | |
| Example | 2b | Myristyl | 1-(acrylamidepropyl) | 1-ethyl-3-hydroxy- | 10 | 80 | 5 | 5 | IPA | 50 |
| 46 | | sulfate | trimethylammonium | ethylimidazolium | | | | | | |
| | | | | bromide | | | | | | |
| Example | 2c | Decyl sulfate | 1-(acrylamidepropyl) | 1-ethyl-3- | 20 | 70 | 5 | 5 | IPA | 60 |
| 47 | | | trimethylammonium | methylimidazolium | | | | | | |
| | | | | thiocyanate 4) | | | | | | |
| Example | 2c | Decyl sulfate | 1-(acrylamidepropyl) | 1-ethyl-3- | 10 | 82 | 3 | 5 | IPA | 50 |
| 48 | | | trimethylammonium | methylimidazolium | | | | | | |
| | | | | thiocyanate | | | | | | |
| Example | 2e | Decyl sulfate | Diallyldimethyl | 1-ethyl-3- | 20 | 70 | 5 | 5 | IPA | 50 |
| 49 | | | ammonium | methylimidazolium | | | | | | |
| | | | | chloride 4) | | | | | | |
| Example | 2d | 2-hexyldecyl | 1-(acrylamidepropyl) | N-ethyl-3- | 20 | 70 | 5 | 5 | IPA | 50 |
| 50 | | sulfate | trimethylammonium | methylpyridium | | | | | | |
| | | | | ethyl sulfate 4) | | | | | | |
| Example | 2f | Lauryl sulfate | Dimethylallylamine | Trihexyl- | 20 | 70 | 5 | 5 | IPA | 50 |
| 51 | | | | (tetradecyl)- | | | | | | |
| | | | | phosphonium | | | | | | |
| | | | | bis(trifluoro- | | | | | | |
| | | | | methylsulfonyl)- | | | | | | |
| | | | | imide 4) | | | | | | |
| Example | 2k | Octyl sulfate | 1-(acrylamidepropyl) | 1-ethyl-3-methyl | 20 | 70 | 5 | 5 | IPA | 45 |
| 52 | | | trimethylammonium | imidazolium bromide | | | | | | |
| Example | 2l | Palmityl | Diallyldimethyl | 1-ethyl-3-methyl | 10 | 80 | 5 | 5 | IPA | 45 |
| 53 | | sulfate | ammonium | imidazolium ethyl | | | | | | |
| | | | | sulfate | | | | | | |

1) IPA: Isopropyl alcohol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 17

| | Coating composition | | | | | | | | | |
|---|---|---|---|---|---|---|---|---|---|---|
| | | | | | Mixing amount | | | | | |
| | | | | | (parts by weight) | | | | | |
| | | | | | Resin | | | | | Solid |
| | | | | | modifier | | | | | component |
| | Resin modifier | | | | (Active | | Ionic | Curing | Organic | (% by |
| | Kind | Anion moiety | Cation moiety | Ionic liquid | component) | DPHA 5) | liquid | agent 3) | solvent | weight) |
| Comparative | — | — | — | — | 0 | 95 | 0 | 5 | IPA 1) | 50 |
| Example 30 | | | | | | | | | | |
| Comparative | 2g | Lauryl sulfate | Triethanolamine | 1-ethyl-3- | 20 | 70 | 5 | 5 | IPA | 60 |
| Example 31 | | | | methylimidazolium | | | | | | |
| | | | | ethyl sulfate 4) | | | | | | |
| Comparative | 2h | Paratoluene- | 1- | 1-ethyl-3- | 20 | 70 | 5 | 5 | IPA | 60 |
| Example 32 | | sulfonic | (acrylamidepropyl) | hydroxyethyl | | | | | | |
| | | acid | trimethylammonium | imidazolium | | | | | | |
| | | | | bromide | | | | | | |
| Comparative | 2i | Methane- | 1- | 1-ethyl-3- | 10 | 82 | 3 | 5 | MeOH 2) | 60 |
| Example 33 | | sulfonic | (acrylamidepropyl) | methylimidazolium | | | | | | |
| | | acid | trimethylammonium | thiocyanate 4) | | | | | | |
| Comparative | 2i | Methane- | 1- | 1-ethyl-3- | 20 | 70 | 5 | 5 | MeOH | 50 |
| Example 34 | | sulfonic | (acrylamidepropyl) | methylimidazolium | | | | | | |
| | | acid | trimethylammonium | thiocyanate | | | | | | |
| Comparative | 2j | Methane- | Diallyldimethyl | 1-ethyl-3- | 20 | 70 | 5 | 5 | IPA | 60 |
| Example 35 | | sulfonic | ammonium | methylimidazolium | | | | | | |
| | | acid | | chloride 4) | | | | | | |
| Comparative | — | — | — | 1-ethyl-3- | 0 | 90 | 5 | 5 | IPA | 60 |
| Example 36 | | | | methylimidazolium | | | | | | |
| | | | | ethyl sulfate | | | | | | |

1) IPA: Isopropyl alcohol
2) MeOH: Methanol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 18

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Example 44 | 70 | 1 | 2.1E+08 | 1.2E+10 | 1.6E+10 | 0.3 |
| Example 45 | 70 | 1 | 6.9E+08 | 3.3E+10 | 4.0E+10 | 0.2 |
| Example 46 | 70 | 1 | 3.2E+08 | 6.3E+10 | 7.9E+10 | 0.4 |
| Example 47 | 70 | 1 | 1.5E+08 | 8.6E+09 | 6.3E+09 | 0.3 |
| Example 48 | 70 | 1 | 6.3E+08 | 1.9E+10 | 2.0E+10 | 0.2 |
| Example 49 | 100 | 2 | 1.9E+09 | 8.9E+10 | 1.6E+11 | 0.3 |
| Example 50 | 70 | 1 | 2.1E+09 | 1.3E+11 | 2.5E+11 | 0.3 |
| Example 51 | 100 | 2 | 3.6E+09 | 2.3E+11 | 4.0E+11 | 0.3 |
| Example 52 | 70 | 1 | 2.1E+09 | 9.8E+10 | 5.0E+10 | 0.2 |
| Example 53 | 70 | 1 | 6.3E+09 | 4.0E+10 | 7.9E+10 | 0.3 |

TABLE 19

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Comparative | 100 | 2 | >1.0E+16 | >1.0E+16 | >1.0E+16 | 0.2 |
| Example 30 | | | | | | |
| Comparative | 70 | 1 | 6.7E+09 | 1.0E+13 | >1.0E+16 | 13.6 |
| Example 31 | | | | | | |
| Comparative | 70 | 1 | 3.2E+09 | 8.3E+10 | 3.2E+12 | 3.2 |
| Example 32 | | | | | | |
| Comparative | 70 | 1 | 4.5E+09 | 2.3E+11 | 6.3E+13 | 10.2 |
| Example 33 | | | | | | |
| Comparative | 100 | 2 | 3.0E+09 | 6.0E+11 | 1.3E+14 | 4.9 |
| Example 34 | | | | | | |
| Comparative | 70 | 1 | 2.9E+09 | 3.0E+11 | 1.6E+11 | 16.2 |
| Example 35 | | | | | | |
| Comparative | 70 | 1 | >1.0E+16 | >1.0E+16 | >1.0E+16 | 0.2 |
| Example 36 | | | | | | |

TABLE 20

| | Coating composition | | | | | | | | | |
|---|---|---|---|---|---|---|---|---|---|---|
| | | | | | Mixing amount (parts by weight) | | | | | |
| | | | | | Resin | | | | | Solid |
| | Resin modifier | | | | modifier | | | | | component |
| | | Anion | | Conductive | (Active | | Conductive | Curing | Organic | (% by |
| | Kind | moiety | Cation moiety | polymer | component) | DPHA 3) | polymer | agent 2) | solvent | weight) |
| Example 54 | 2l | Palmityl | Diallyldimethylammonium | PED-E60 4) | 10 | 84 | 1 | 5 | IPA 1) | 44 |
| | | sulfate | | | | | | | | |
| Example 55 | 2c | Decyl | 1-(acrylamidepropyl) | Panipol-T 5) | 10 | 89.5 | 0.5 | 5 | IPA | 37 |
| | | sulfate | trimethylammonium | | | | | | | |
| Comparative | — | — | — | PED-E60 | 0 | 94 | 1 | 5 | IPA | 43 |
| Example 37 | | | | | | | | | | |
| Comparative | — | — | — | PED-E60 | 0 | 90 | 5 | 5 | IPA | 29 |
| Example 38 | | | | | | | | | | |
| Comparative | — | — | — | Panipol-T | 0 | 90 | 5 | 5 | IPA | 42 |
| Example 39 | | | | | | | | | | |

1) IPA: Isopropyl alcohol
2) Curing agent: Irgacure 184 (manufactured by Ciba Specialty Chemicals Inc.)
3) DPHA: Dipentaerythritol hexaacrylate
4) PED-E60: Polythiophene manufactured by Polymerits Corp.
5) Panipol-T: Polyaniline manufactured by PANIPOL Oy

TABLE 21

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Example 54 | 70 | 1 | 9.1E+09 | 8.0E+10 | 1.0E+11 | 0.4 |
| Example 55 | 70 | 1 | 3.0E+10 | 1.0E+11 | 8.0E+10 | 0.5 |
| Comparative | 70 | 1 | 8.4E+15 | >1.0E+16 | >1.0E+16 | 0.4 |
| Example 37 | | | | | | |
| Comparative | 70 | 1 | 3.4E+11 | 1.9E+12 | 3.2E+12 | 1.0 |
| Example 38 | | | | | | |
| Comparative | 70 | 1 | >1.0E+16 | >1.0E+16 | >1.0E+16 | 1.0 |
| Example 39 | | | | | | |

TABLE 22

| | Coating composition | | | | | | | |
|---|---|---|---|---|---|---|---|---|
| | | | | Mixing amount (parts by weight) | | | | |
| | | | | Resin | | | | |
| | Resin modifier | | | modifier | | | | Solid |
| | | Anion | | active | | Curing | Organic | component |
| | Kind | moiety | Cation moiety | component) | DPHA 4) | agent 3) | solvent | (% by weight) |
| Example | 3a | Decane | 1-(acrylamidepropyl) | 20 | 75 | 5 | IPA 1) | 60 |
| 56 | | sulfonate | trimethylammonium | | | | | |
| Example | 3a | Decane | 1-(acrylamidepropyl) | 10 | 85 | 5 | IPA | 60 |
| 57 | | sulfonate | trimethylammonium | | | | | |
| Example | 3a | Decane | 1-(acrylamidepropyl) | 5 | 90 | 5 | IPA | 60 |
| 58 | | sulfonate | trimethylammonium | | | | | |
| Example | 3a | Decane | 1-(acrylamidepropyl) | 3 | 92 | 5 | IPA | 60 |
| 59 | | sulfonate | trimethylammonium | | | | | |
| Example | 3b | Pentadecane | 1-(acrylamidepropyl) | 20 | 75 | 5 | IPA | 60 |
| 60 | | sulfonate | trimethylammonium | | | | | |
| Example | 3b | Pentadecane | 1-(acrylamidepropyl) | 5 | 90 | 5 | IPA | 60 |
| 61 | | sulfonate | trimethylammonium | | | | | |
| Example | 3c | Decane | Diallyldimethyl | 20 | 75 | 5 | IPA | 60 |
| 62 | | sulfonate | ammonium | | | | | |

| | Drying conditions | | Evaluation of coating film | | |
|---|---|---|---|---|---|
| | of coating film | | Antistatic | Water | |
| | Temperature | Time | properties | resistance | Transparency |
| | (° C.) | (minute) | (Ω) | (log Ω) | (%) |
| Example | 70 | 1 | 1.3E+09 | 1.6E+09 | 0.1 |
| 56 | | | | | |
| Example | 70 | 1 | 4.0E+09 | 1.3E+09 | 0.2 |
| 57 | | | | | |
| Example | 70 | 1 | 2.5E+10 | 1.6E+11 | 0.2 |
| 58 | | | | | |
| Example | 70 | 1 | 2.0E+11 | 6.3E+11 | 0.2 |
| 59 | | | | | |
| Example | 70 | 1 | 1.3E+10 | 2.0E+10 | 0.2 |
| 60 | | | | | |
| Example | 70 | 1 | 1.6E+10 | 1.0E+11 | 0.2 |
| 61 | | | | | |
| Example | 70 | 1 | 2.0E+09 | 2.5E+09 | 0.2 |
| 62 | | | | | |

1) IPA: Isopropyl alcohol
3) Curing agent: Irgacure 184 (manufactured by Ciba Specialty Chemicals Inc.)
4) DPHA: Dipentaerythritol hexaacrylate

TABLE 23

| | Coating composition | | | | | | | |
|---|---|---|---|---|---|---|---|---|
| | | | | Mixing amount | | | | |
| | | | | (parts by weight) | | | | |
| | | | | Resin | | | | Solid |
| | | | | modifier | | | | component |
| | Resin modifier | | | (active | | Curing | Organic | (% by |
| | Kind | Anion moiety | Cation moiety | component) | DPHA 4) | agent 3) | solvent | weight) |
| Comparative | — | — | — | 0 | 95 | 5 | IPA 1) | 50 |
| Example 40 | | | | | | | | |
| Comparative | 3d | Paratoluenesulfonic | 1-(acrylamidepropyl) | 20 | 75 | 5 | IPA | 60 |
| Example 41 | | acid | trimethylammonium | | | | | |
| Comparative | 3e | Methanesulfonic | 1-(acrylamidepropyl) | 20 | 75 | 5 | MeOH 2) | 60 |
| Example 42 | | acid | trimethylammonium | | | | | |
| Comparative | 3e | Methanesulfonic | 1-(acrylamidepropyl) | 5 | 90 | 5 | MeOH | 50 |
| Example 43 | | acid | trimethylammonium | | | | | |
| Comparative | 3f | Methanesulfonic | Diallyldimethyl | 20 | 75 | 5 | IPA | 60 |
| Example 44 | | acid | ammonium | | | | | |

| | Drying conditions | | Evaluation of coating film | | |
|---|---|---|---|---|---|
| | of coating film | | Antistatic | Water | |
| | Temperature | Time | properties | resistance | Transparency |
| | (° C.) | (minute) | (Ω) | (log Ω) | (%) |
| Comparative | 70 | 1 | >1.0E+16 | >1.0E+16 | 0.2 |
| Example 40 | | | | | |
| Comparative | 70 | 1 | 6.3E+11 | 3.2E+12 | 3.0 |
| Example 41 | | | | | |
| Comparative | 70 | 1 | 6.3E+12 | 6.3E+13 | 9.2 |
| Example 42 | | | | | |
| Comparative | 70 | 1 | 3.2E+14 | 4.0E+14 | 7.0 |
| Example 43 | | | | | |
| Comparative | 70 | 1 | 1.6E+10 | 1.6E+11 | 15.0 |
| Example 44 | | | | | |

1) IPA: Isopropyl alcohol
2) MeOH: Methanol
3) Curing agent: Irgacure 184 (manufactured by Ciba Specialty Chemicals Inc.)
4) DPHA: Dipentaerythritol hexaacrylate

TABLE 24

| | Coating composition | | | | | | | | | | |
|---|---|---|---|---|---|---|---|---|---|---|---|
| | | | | Mixing amount | | | | | | | |
| | | | | (parts by weight) | | | | | | | |
| | | | | Resin | | | | Solid | Drying conditions | | |
| | Resin modifier solution | | | modifier | | | | component | of coating film | | Contact |
| | | Anion | | (active | | Curing | Organic | (% by | Temperature | Time | angle |
| | Kind | moiety | Cation moiety | component) | DPHA 4) | agent 3) | solvent | weight) | (° C.) | (minute) | (degree) |
| Example 63 | 3a | Decane | 1-(acrylamide- | 20 | 75 | 5 | IPA 1) | 60 | 70 | 1 | 24 |
| | | sulfonate | propyl) | | | | | | | | |
| | | | trimethyl- | | | | | | | | |
| | | | ammonium | | | | | | | | |
| Example 64 | 3b | Pentadecane | 1-(acrylamide- | 20 | 75 | 5 | IPA | 60 | 70 | 1 | 22 |
| | | sulfonate | propyl) | | | | | | | | |
| | | | trimethyl- | | | | | | | | |
| | | | ammonium | | | | | | | | |
| Example 65 | 3c | Decane | Diallyldimethyl | 20 | 75 | 5 | IPA | 60 | 70 | 1 | 29 |
| | | sulfonate | ammonium | | | | | | | | |
| Comparative | — | — | — | 0 | 95 | 5 | IPA | 60 | 70 | 1 | 38 |
| Example 45 | | | | | | | | | | | |
| Comparative | 3e | Methane- | 1-(acrylamide- | 20 | 75 | 5 | IPA | 60 | 70 | 1 | 39 |
| Example 46 | | sulfonic | propyl) | | | | | | | | |
| | | acid | trimethyl- | | | | | | | | |
| | | | ammonium | | | | | | | | |
| Comparative | 3d | Paratoluene- | 1-(acrylamide- | 20 | 75 | 5 | IPA | 60 | 70 | 1 | 38 |
| Example 47 | | sulfonic | propyl) | | | | | | | | |
| | | acid | trimethyl- | | | | | | | | |
| | | | ammonium | | | | | | | | |

1) IPA: Isopropyl alcohol
3) Curing agent: Irgacure 184 (manufactured by Ciba Specialty Chemicals Inc.)
4) DPHA: Dipentaerythritol hexaacrylate

TABLE 25

| | Coating composition | | | | | | | | | |
|---|---|---|---|---|---|---|---|---|---|---|
| | | | | | Mixing amount | | | | | |
| | | | | | (parts by weight) | | | | | |
| | | | | | Resin | | | | | Solid |
| | Resin modifier | | | | modifier | | | | | component |
| | | Anion | | | (Active | | Ionic | Curing | Organic | (% by |
| | Kind | moiety | Cation moiety | Ionic liquid | component) | DPHA 5) | liquid | agent 3) | solvent | weight) |
| Example | 3a | Decane | 1-(acrylamidepropyl) | 1-ethyl-3- | 20 | 70 | 5 | 5 | IPA 1) | 60 |
| 66 | | sulfonate | trimethylammonium | methylimidazolium | | | | | | |
| | | | | ethyl sulfate 4) | | | | | | |
| Example | 3a | Decane | 1-(acrylamidepropyl) | 1-ethyl-3- | 10 | 80 | 5 | 5 | IPA | 60 |
| 67 | | sulfonate | trimethylammonium | methylimidazolium | | | | | | |
| | | | | ethyl sulfate | | | | | | |
| Example | 3a | Decane | 1-(acrylamidepropyl) | 1-ethyl-3- | 5 | 85 | 5 | 5 | IPA | 60 |
| 68 | | sulfonate | trimethylammonium | methylimidazolium | | | | | | |
| | | | | thiocyanate 4) | | | | | | |
| Example | 3a | Decane | 1-(acrylamidepropyl) | 1-ethyl-3- | 3 | 89 | 3 | 5 | IPA | 60 |
| 69 | | sulfonate | trimethylammonium | methylimidazolium | | | | | | |
| | | | | thiocyanate | | | | | | |
| Example | 3b | Pentadecane | 1-(acrylamidepropyl) | N-ethyl-3- | 20 | 70 | 5 | 5 | IPA | 60 |
| 70 | | sulfonate | trimethylammonium | methylpyridium | | | | | | |
| | | | | ethyl sulfate 4) | | | | | | |
| Example | 3b | Pentadecane | 1-(acrylamidepropyl) | 1-ethyl-3- | 5 | 85 | 5 | 5 | IPA | 60 |
| 71 | | sulfonate | trimethylammonium | methylimidazolium | | | | | | |
| | | | | chloride 4) | | | | | | |
| Example | 3c | Decane | Diallyldimethyl | 1-ethyl-3- | 20 | 72 | 3 | 5 | IPA | 60 |
| 72 | | sulfonate | ammonium | hydroxyethyl- | | | | | | |
| | | | | imidazolium | | | | | | |
| | | | | bromide | | | | | | |

1) IPA: Isopropyl alcohol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 26

| | Coating composition | | | | | | | | | |
|---|---|---|---|---|---|---|---|---|---|---|
| | | | | | Mixing amount | | | | | |
| | | | | | (parts by weight) | | | | | |
| | | | | | Resin | | | | | Solid |
| | Resin modifier | | | | modifier | | | | | component |
| | | Anion | | | (Active | | Ionic | Curing | Organic | (% by |
| | Kind | moiety | Cation moiety | Ionic liquid | component) | DPHA 5) | liquid | agent 3) | solvent | weight) |
| Comparative | — | — | — | — | 0 | 95 | 0 | 5 | IPA 1) | 50 |
| Example 48 | | | | | | | | | | |
| Comparative | 3d | Paratoluene- | 1-(acrylamidepropyl) | 1-ethyl-3- | 20 | 70 | 5 | 5 | IPA | 60 |
| Example 49 | | sulfonic acid | trimethylammonium | methylimidazolium | | | | | | |
| | | | | ethyl sulfate 4) | | | | | | |
| Comparative | 3e | Methane- | 1-(acrylamidepropyl) | 1-ethyl-3- | 10 | 80 | 5 | 5 | MeOH 2) | 50 |
| Example 50 | | sulfonic acid | trimethylammonium | methylimidazolium | | | | | | |
| | | | | ethyl sulfate | | | | | | |
| Comparative | 3e | Methane- | 1-(acrylamidepropyl) | 1-ethyl-3- | 20 | 70 | 5 | 5 | MeOH | 50 |
| Example 51 | | sulfonic acid | trimethylammonium | methylimidazolium | | | | | | |
| | | | | thiocyanate 4) | | | | | | |
| Comparative | 3f | Methane- | Diallyldimethyl | 1-ethyl-3- | 20 | 70 | 5 | 5 | IPA | 60 |
| Example 52 | | sulfonic acid | ammonium | methylimidazolium | | | | | | |
| | | | | chloride 4) | | | | | | |
| Comparative | — | — | — | 1-ethyl-3- | 0 | 90 | 5 | 5 | IPA | 60 |
| Example 53 | | | | methylimidazolium | | | | | | |
| | | | | ethyl sulfate | | | | | | |

1) IPA: Isopropyl alcohol
2) MeOH: Methanol
3) Curing agent: Irgacure 184 (manufactured by Ciba Specialty Chemicals Inc.)
4) Reagent manufactured by Merck & Co., Inc.
5) DPHA: Dipentaerythritol hexaacrylate

TABLE 27

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (log Ω) | (%) |
| Example 66 | 70 | 1 | 4.5E+08 | 9.7E+09 | 1.6E+09 | 0.2 |
| Example 67 | 70 | 1 | 6.8E+08 | 1.3E+10 | 1.3E+09 | 0.2 |
| Example 68 | 70 | 1 | 2.0E+09 | 5.4E+10 | 1.6E+11 | 0.2 |
| Example 69 | 70 | 1 | 1.6E+10 | 1.3E+11 | 6.3E+11 | 0.2 |
| Example 70 | 70 | 1 | 1.0E+09 | 2.3E+10 | 2.0E+10 | 0.2 |
| Example 71 | 70 | 1 | 9.7E+09 | 6.8E+10 | 1.0E+11 | 0.2 |
| Example 72 | 70 | 1 | 2.2E+09 | 3.7E+10 | 2.5E+09 | 0.2 |

TABLE 28

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (log Ω) | (%) |
| Comparative | 70 | 1 | >1.0E+16 | >1.0E+16 | >1.0E+16 | 0.2 |
| Example 48 | | | | | | |
| Comparative | 70 | 1 | 6.0E+09 | 3.0E+10 | 3.2E+12 | 3.0 |
| Example 49 | | | | | | |
| Comparative | 100 | 2 | 3.0E+09 | 6.0E+11 | 1.3E+14 | 4.9 |
| Example 50 | | | | | | |
| Comparative | 100 | 2 | 3.0E+09 | 6.0E+11 | 1.3E+14 | 4.9 |
| Example 51 | | | | | | |
| Comparative | 70 | 1 | 2.9E+09 | 3.0E+11 | 1.6E+11 | 16.2 |
| Example 52 | | | | | | |
| Comparative | 70 | 1 | >1.0E+16 | >1.0E+16 | >1.0E+16 | 0.2 |
| Example 53 | | | | | | |

TABLE 29

| | Coating composition | | | | | | | | | |
|---|---|---|---|---|---|---|---|---|---|---|
| | | | | | Mixing amount (parts by weight) | | | | | |
| | | | | | Resin | | | | | Solid |
| | Resin modifier | | | | modifier | | | | | component |
| | | Anion | | Conductive | (Active | | Conductive | Curing | Organic | (% by |
| | Kind | moiety | Cation moiety | polymer | component) | DPHA 3) | polymer | agent 2) | solvent | weight) |
| Example 73 | 3a | Decane | 1-(acrylamidepropyl) | PED-E60 4) | 10 | 84 | 1 | 5 | IPA 1) | 42 |
| | | sulfonate | trimethylammonium | | | | | | | |
| Comparative | — | — | — | PED-E60 | 0 | 94 | 1 | 5 | IPA | 43 |
| Example 54 | | | | | | | | | | |
| Comparative | — | — | — | PED-E60 | 0 | 90 | 5 | 5 | IPA | 29 |
| Example 55 | | | | | | | | | | |

1) IPA: Isopropyl alcohol
2) Curing agent: Irgacure 184 (manufactured by Ciba Specialty Chemicals Inc.)
3) DPHA: Dipentaerythritol hexaacrylate
4) PED-E60: Polythiophene manufactured by Polymerits Corp.

TABLE 30

| | | | Evaluation of coating film | | | |
|---|---|---|---|---|---|---|
| | | | | Low- | | |
| | Drying conditions | | | humidity | | |
| | of coating film | | Antistatic | antistatic | Water | |
| | Temperature | Time | properties | properties | resistance | Transparency |
| | (° C.) | (minute) | (Ω) | (Ω) | (Ω) | (%) |
| Example 73 | 70 | 1 | 8.0E+09 | 9.0E+10 | 1.1E+11 | 0.4 |
| Comparative | 70 | 1 | 8.4E+15 | >1.0E+16 | >1.0E+16 | 0.4 |
| Example 54 | | | | | | |
| Comparative | 70 | 1 | 3.4E+11 | 1.9E+12 | 3.2E+12 | 1.0 |
| Example 55 | | | | | | |

1-11. (canceled)

12. A method for producing a coating film, which comprises coating a coating composition comprising a resin modifier represented by any one of the following formulas (Ia), (Ib) and (Ic) and an organic solvent on a base material, and then forming a coating film on the base material:
R³¹—O-(AO)ₙ—SO₃⁻B⁺ (Ia)
wherein R³¹ represents a hydrocarbon group having 1 to 22 carbon atoms, AO represents an alkylene oxide group having 2 to 4 carbon atoms, n represents an average addition molar number of AO and is a positive number of 100 or less, and B⁺ represents an ammonium ion (C) having a polymerizable unsaturated group;
R³²—OSO₃⁻B⁺ (Ib)
wherein R³² represents a hydrocarbon group having 6 to 22 carbon atoms, and B+ represents an ammonium ion (C) having a polymerizable unsaturated group;
R³³—SO₃⁻B⁺ (Ic)
wherein R³³ represents a hydrocarbon group having 8 to 22 carbon atoms, and B+ represents ammonium ion (C) having a polymerizable unsaturated group.

13. The method for producing a coating film according to claim 12, wherein the coating film is formed on the base material by irradiating the coating composition with active energy rays.

14. The method for producing a coating film according to claim 12, wherein in the formulas (Ia), (Ib) and (Ic), R³¹, R³² and R³³ each represents a hydrocarbon group having 8 to 20 carbon atoms.

15. The method for producing a coating film according to claim 12, wherein in the formula (Ia), n represents 1 to 30.

16. The method for producing a coating film according to claim 12, wherein the ammonium ion (C) is represented by the following formula (II) or the following formula (III): 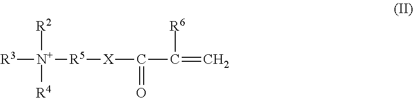
wherein R², R³, R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH; and 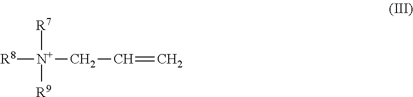
wherein R⁷ and R⁸ each independently represents a hydrogen atom or a hydrocarbon group having 1 to 8 carbon atoms, and R⁹ represents a hydrocarbon group having 1 to 8 carbon atoms.

17. The method for producing a coating film according to claim 16, wherein in the formula (II), R², R³, R⁴ each independently represents a hydrocarbon group having 1 or 2 carbon atoms.

18. The method for producing a coating film according to claim 16, wherein in the formula (II), R⁵ represents an alkylene group having 2 or 3 carbon atoms.

19. The method for producing a coating film according to claim 16, wherein in the formula (III), R⁷, R⁸, R⁹ each independently represents a hydrocarbon group having 1 to 3 carbon atoms.

20. The method for producing a coating film according to claim 16, wherein in the formula (III), R⁷ is an allyl group and R⁸ is a hydrocarbon group having 1 to 3 carbon atoms.

21. The method for producing a coating film according to claim 16, wherein in the formula (III), R⁹ represents a hydrocarbon group having 1 or 2 carbon atoms.

22. The method for producing a coating film according to claim 16, wherein the resin modifier is represented by the formula (Ia) and the ammonium ion (C) is represented by the formula (II).

23. The method for producing a coating film according to claim 16, wherein the resin modifier is represented by the formula (Ia) and the ammonium ion (C) is represented by the formula (III).

24. The method for producing a coating film according to claim 12, wherein the coating composition further contains a resin or resin monomer capable of reacting by irradiation with active energy rays and the coating film is formed on the base material by irradiating the coating composition with active energy rays.

25. The method for producing a coating film according to claim 24, wherein the content of the resin modifier is 0.5 to 50% by weight, the content of the resin or resin monomer capable of reacting by irradiation with active energy rays is 20 to 80% by weight, and the content of the organic solvent is 10 to 70% by weight.

26. The method for producing a coating film according to claim 24, wherein the amount of the resin modifier is 1 to 50 parts by weight based on 100 parts by weight of the resin or resin monomer capable of reacting by irradiation with active energy rays.

27. The method for producing a coating film according to claim 12, which further contains an ionic liquid.

28. The method for producing a coating film according to claim 12, which further contains a conductive polymer.

29. An antistatic or anti-fogging coating film obtained by the production method according to claim 12.

30. The method for producing a coating film comprising structures represented by any one of the following formulas (IVa), (IVb), (IVc), (Va), (Vb) and (Vc) in at least one portion: 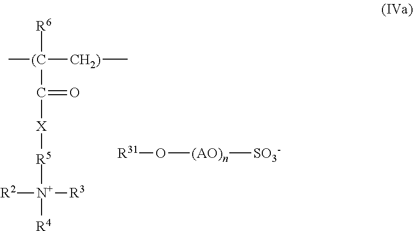
wherein R³¹ represents a hydrocarbon group having 1 to 22 carbon atoms, AO represents an alkylene oxide group having 2 to 4 carbon atoms, n is an average addition molar number of AO and is a positive number of 100 or less, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH; 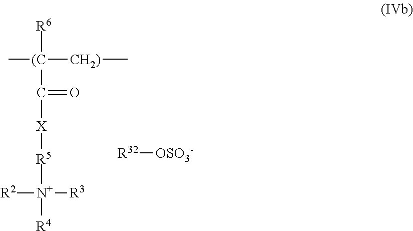
wherein R³² represents a hydrocarbon group having 6 to 22 carbon atoms, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH; 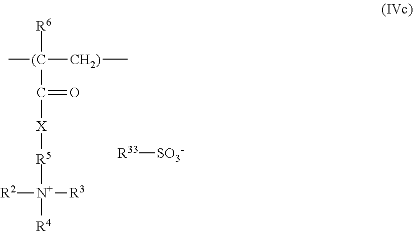
wherein R³³ represents a hydrocarbon group having 8 to 22 carbon atoms, R², R³ and R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH; 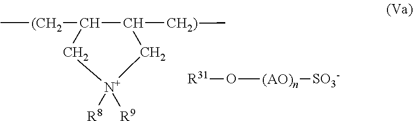
wherein R³¹ represents a hydrocarbon group having 1 to 22 carbon atoms, AO represents an alkylene oxide group having 2 to 4 carbon atoms, n represents an average addition molar number of AO and is a positive number of 100 or less, R⁸ represents a hydrogen atom or a hydrocarbon group having 1 to 8 carbon atoms, and R⁹ represents a hydrocarbon group having 1 to 8 carbon atoms; 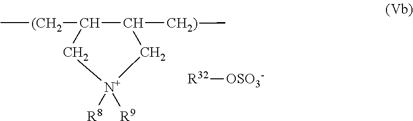
wherein R³² represents a hydrocarbon group having 6 to 22 carbon atoms, R⁸ represents a hydrogen atom or a hydrocarbon group having 1 to 8 carbon atoms, and R⁹ represents a hydrocarbon group having 1 to 8 carbon atoms; and 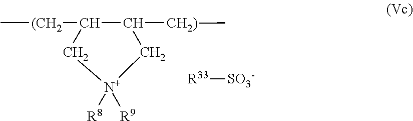
wherein R³³ represents a hydrocarbon group having 8 to 22 carbon atoms, R⁸ represents a hydrogen atom or a hydrocarbon group having 1 to 8 carbon atoms, and R⁹ represents a hydrocarbon group having 1 to 8 carbon atoms.

31. An antistatic or anti-fogging method using a resin modifier represented by any one of the following formulas (Ia), (Ib) and (Ic):
R³¹—O-(AO)ₙ—SO₃⁻B⁺ (Ia)
wherein R³¹ represents a hydrocarbon group having 1 to 22 carbon atoms, AO represents an alkylene oxide group having 2 to 4 carbon atoms, n represents an average addition molar number of AO and is a positive number of 100 or less, and B⁺ represents an ammonium ion (C) having a polymerizable unsaturated group;
R³²—OSO₃⁻B⁺ (Ib)
wherein R³² represents a hydrocarbon group having 6 to 22 carbon atoms, and B+ represents an ammonium ion (C) having a polymerizable unsaturated group;
R³³—SO₃⁻B⁺ (Ic)
wherein R³³ represents a hydrocarbon group having 8 to 22 carbon atoms, and B+ represents ammonium ion (C) having a polymerizable unsaturated group.

32. The antistatic or anti-fogging method according to claim 31, wherein the ammonium ion (C) is represented by the following formula (II) or (III): 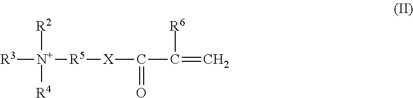
wherein R², R³, R⁴ each independently represents a hydrogen atom or a hydrocarbon group having 1 to 8 carbon atoms, R⁵ represents an alkylene group having 2 to 5 carbon atoms, R⁶ represents a hydrogen atom or a methyl group, and X represents O or NH; and 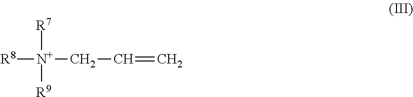
wherein R⁷ and R⁸ each independently represents a hydrogen atom or a hydrocarbon group having 1 to 8 carbon atoms, and R⁹ represents a hydrocarbon group having 1 to 8 carbon atoms.
